(19) United States
(12) Patent Application Publication
(10) Pub. No.: US 2023/0134345 A1
(43) Pub. Date: May 4, 2023

(54) CHIMERIC ANTIGEN RECEPTOR COMPRISING THIRD SIGNAL RECEPTOR AND USE THEREOF

(71) Applicant: SHANGHAI LONGYAO BIOTECHNOLOGY INC., LTD., Shanghai (CN)

(72) Inventors: Xuanming YANG, Shanghai (CN); Yangxin FU, Shanghai (CN); Xin WANG, Shanghai (CN); Shengqin YE, Shanghai (CN); Min LI, Shanghai (CN)

(21) Appl. No.: 18/047,750

(22) Filed: Oct. 19, 2022

Related U.S. Application Data
(63) Continuation-in-part of application No. 17/127,283, filed on Dec. 18, 2020, now Pat. No. 11,524,034.
Application No. 17/127,283 is a continuation of application No. PCT/CN2019/077923, filed on Mar. 13, 2019.

(30) Foreign Application Priority Data
Jun. 20, 2018 (CN) 201810636414.1

(51) Int. Cl. C07K 16/30; A61P 35/00

(52) U.S. Cl. CPC C07K 16/30; A61P 35/00; C07K 2317/622; C07K 2319/02; C07K 2319/03; C07K 2317/53

(57) ABSTRACT

The present invention relates a chimeric antigen receptor, which has a structure of X-Y-CD3zeta-M-N; wherein X comprises a tumor targeting antibody or a ligand or receptor capable of specifically binding to a tumor. Y is an intracellular region of a costimulatory receptor selected from ICOS, CD28, CD27, HVEM, LIGHT, CD40L, 4-1BB, OX40, DR3, GITR, CD30, TIM1, SLAM, CD2, and CD226; M is an intracellular region of a gamma chain family cytokine receptor, the cytokine receptor being selected from IL2Ra, IL2Rb, IL4Ra, IL7Ra, IL9Ra, IL15Ra, and IL21Ra. N is an intracellular region of IL2Rg. The present invention further provides a CAR-T cell constructed from the recombinant expression vector of said chimeric antigen receptor, a preparation method therefor and the use thereof. The CAR-T cell of the present invention significantly improves tumor killing capacity and amplification capacity. The CAR T cell comprises a third signal receptor, has a potential effect-enhancing function, and only works on the CAR-T cell, thereby reducing the risk of causing an immune side effect.

The present application is a continuation-in-part of U.S. patent application Ser. No. 17/127,283, filed Dec. 18, 2020, a continuation of PCT/CN2019/077923, filed Mar. 13, 2019, which claims the benefit of CN application 201810636414.1 (CN), filed Jun. 20, 2018, all of which are hereby incorporated by reference in their entireties herein.

### SEQUENCE LISTING

This application incorporates in its entirety the Sequence Listing entitled “271302-517374.xml” (52,067 bytes), which was created on Oct. 13, 2022, and filed electronically herewith.

### TECHNICAL FIELD

The present invention relates to the field of cellular immunotherapy, especially to a chimeric antigen receptor comprising a third signal receptor and use thereof.

### BACKGROUND OF THE INVENTION

The use of immunological therapy for overcoming tumors has always been an important direction in the application of immunology in translational medicine. With the development of various omics (genomics, proteomics, etc.), tumor cells have been widely recognized due to their immunogenicity caused by mutations, which lays a theoretical foundation for tumor immunotherapy. At the same time, with the accumulation of tumor immunology research itself, tumor immunotherapy has recently made a great progress, and a series of new immunotherapy methods have gradually entered into the clinic. The current tumor immunology research has established the central position of T cell killing in tumor immunotherapy, and the chimeric antigen receptor T cell (CAR-T cell) is a tumor-killing cell which has combined the targeted recognition of antibody and the tumor-killing function of T cell, and generated by artificial modification.

The concept of chimeric antigen receptor T cell was first proposed by Gross, Waks and Eshhar in 1989. They expressed TNP-recognizing antibodies on T cells, achieving the antigen-specific, non-MHC-restricted activation of T cell and enhancement of effect, and proposed the concept of applying the CAR-T technology in tumor treatment. According to this principle, tumor-specific antibodies are embedded into T cells, which will give T cells new tumor-killing capabilities. After that, the CAR-T technology was introduced into anti-tumor clinical trials, but early CAR-T cells are not ideal in final clinical results since their intracellular signaling domain contains only the first signal, and the selected tumor type is a solid tumor. In 2008, the Fred Hutchison Cancer Institute and other institutions used CAR-T to treat B cell lymphoma, although the treatment results were not ideal, the key to this clinical trial is to confirm that the CAR-T treatment with CD20-expressing B cells as the target is relatively safe. Subsequently, in 2010, NCI reported a case of successful treatment of B cell lymphoma, using CAR-T targeting CD19, the patient's lymphoma was controlled, normal B cells were also eliminated, and serum Ig was significantly reduced, providing a theoretical and practical support for the effectiveness of CAR-T in the treatment of B cell-derived lymphomas. In 2011, a team led by Dr. Carl June of the University of Pennsylvania in the United States used CAR-T that specifically recognizes CD19 for the treatment of chronic lymphocytic leukemia derived from B cells, showing a “cure” effect. Clinical trials have been launched in relapsed and refractory acute lymphoblastic cell leukemia, and good results have also been achieved. Due to this breakthrough progress and the development of other immune regulation methods, Science magazine ranked tumor immunotherapy as the number one scientific and technological breakthrough in 2013. This success has caused widespread influence in countries around the world, and countries have begun to carry out a large number of CAR-T-based scientific research and clinical trials of tumor treatment.

The structure of CAR consists of an extracellular antigen recognition domain, an extracellular hinge region, a transmembrane domain, and an intracellular signal transduction domain. The extracellular antigen recognition domain consists commonly of a single-chain antibody, which specifically recognizes a tumor cell membrane surface molecule, or can be a ligand or a receptor of certain tumor-specific antigens. The extracellular hinge region is a spatial structure that separates the antigen recognition domain from the transmembrane domain, and its purpose is to provide a suitable spatial position, so that the extracellular antigen recognition domain can maintain the correct structure and transmit the intracellular signals before and after recognizing the antigen. The transmembrane domain is a structural domain for ensuring the positioning of the CAR molecule on the membrane surface. The intracellular signal transduction domain is a key part of mediating the CAR signal transduction, and is usually a combination of one or several first signals (for the recognition of TCR and MHC-I-peptide complex) and second signals (for the recognition of costimulatory receptor and costimulatory ligand). The first-generation CAR contains only the first signal, the second-generation CAR has one first signal and one second signal, and the third-generation CAR has one first signal and two second signal domains. Although CAR-T has achieved a great success in the treatment of leukemia derived from B cell, its relatively high recurrence rate and low effectiveness for solid tumors are great challenges currently. Therefore, there is an urgent clinic need of developing a new generation of high-efficiency CAR-T currently. In addition to the third-generation CAR-T, there are currently other new CAR-T design strategies, that is, introducing new regulatory molecules independent of CAR on the basis of the second-generation CAR-T to further enhance the function of CAR-T.

The application of CAR-T targeting the B cell surface targeting molecules CD19 and CD20 prepared from the patient's own blood cells in the treatment of B cell leukemia has been relatively mature, but there are a large number of recurrences, even if the response rate is high. In addition, the treatment efficiency for solid lymphoma is relatively low, which is related to the immunosuppressive microenvironment in solid tumors.

In solid tumors, there are a variety of immune cells, tumor cells and stromal cells, which together constitute the tumor microenvironment. The tumor microenvironment is usually immunosuppressive, and can inhibit endogenous anti-tumor T cell responses or adoptive T cells (such as CAR-T) at multiple levels, for example, leading to exhaustion of T cells and loss of tumor killing function, and eventually leading to the clearance of T cells. How to enhance the activation ability of CAR-T in solid tumors so that it can fight against the immune suppression in the tumor microenvironment is an important idea and direction for expanding CAR-T to solid tumor treatment.

However, the current CAR-T domains in clinical use still have insufficient tumor killing and expansion abilities, and have poor efficacy in controlling solid tumors/metastasis. Some CAR-T uses novel regulatory molecules such as IL-12, 4-1BBL, etc. These molecules will also produce non-specific activation effects on other non-CAR-T cells in addition to affecting the CAR-T, which may cause potential immune side effects.

### SUMMARY OF THE INVENTION

An object of the present invention is to address the defects in the prior art, provide a chimeric antigen receptor including a third signal receptor and use thereof, and provide a CAR-T cell constructed by a recombinant expression vector of the chimeric antigen receptor, and in the CAR-T cell, the activation of T cells is regulated by a first signal (for recognizing of the TCR and MHC-I-peptide complex), a second signal (for recognizing of co-stimulating receptor and co-stimulating ligand) and a third signal (for recognizing of cytokine receptor and cytokine), which synergistically achieve massive expansion of T cells, exert effector functions, and eliminate infection or tumors.

To achieve the aforesaid object, the present invention utilizes the following technical solutions:

In an aspect of the invention, the present invention provides a chimeric antigen receptor (CAR) including a third signal receptor, said chimeric antigen receptor comprises a structure of X-Y-CD3zeta-M-N;

wherein, X comprises a tumor-targeting antibody or a ligand or receptor capable of binding to a tumor; Y is an intracellular domain of a costimulatory receptor, said costimulatory receptor is selected from ICOS, CD28, CD27, HVEM, LIGHT, CD40L, 4-1BB, OX40, DR3, GITR, CD30, TIM1, SLAM, CD2, CD226; M is an intracellular domain of a gamma chain family cytokine receptor, said cytokine receptor is selected from IL2Ra, IL2Rb, IL4Ra, IL7Ra, IL9Ra, IL15Ra, IL21Ra; and N is an intracellular domain of IL2Rg.

For further optimizing the aforesaid chimeric antigen receptor, the technical means utilized by the present invention further includes:

further, said X is selected from anti-CD19 antibody, anti-CD20 antibody, anti-EGFR antibody, anti-HER2 antibody, anti-EGFRVIII antibody, anti-PSMA antibody, anti-BCMA antibody, anti-CD22 antibody, anti-CD30 antibody. Understandably, X can also be other protein capable of specifically binding a tumor. For example, X can be anti-CLDN18.2 antibody.

In some embodiments, said antibody comprises scFv.

In some embodiments, said X is selected from anti-CD19 scFv, anti-CD20 scFv, anti-EGFR scFv, anti-HER2 scFv, anti-EGFRVIII scFv, anti-PSMA scFv, anti-BCMA scFv, anti-CD22 scFv, anti-CD30 scFv and anti-CLDN18.2 scFv.

In some embodiments, the sequence of said anti-CD20 scFv is as set forth in SEQ ID No: 1; the sequence of said anti-CD19 scFv is as set forth in SEQ ID No: 2; the sequence of said anti-CLDN18.2 scFv is as set forth in SEQ ID No: 3; and/or the sequence of said anti-EGFR scFv is as set forth in SEQ ID No: 4.

In some embodiments, said Y is an intracellular domain of 4-1BB.

In some embodiments, the sequence of said intracellular domain of IL7Ra is as set forth in SEQ ID No: 8; the sequence of said intracellular domain of IL2Rb is as set forth in SEQ ID No: 9; the sequence of said intracellular domain of IL4Ra is as set forth in SEQ ID No: 10; the sequence of said intracellular domain of IL9Ra is as set forth in SEQ ID No: 11; the sequence of said intracellular domain of IL21Ra is as set forth in SEQ ID No: 12; the sequence of said intracellular domain of IL2Rg is as set forth in SEQ ID No: 13; the sequence of said intracellular domain of 4-1BB is as set forth in SEQ ID No:6; and/or the sequence of said CD3zata is as set forth in SEQ ID No: 7.

In some embodiments, wherein said X is selected from anti-CD19 scFv, anti-CD20 scFv, anti-EGFR scFv, HER2 scFv, EGFRVIII scFv, anti-PSMA scFv, anti-BCMA scFv, anti-CD22 scFv, anti-CD30 scFv and anti-CLDN18.2 scFv;

and said Y is an intracellular domain of 4-1BB, said M is one selected from intracellular domain of IL2Rb, intracellular domain of IL4Ra, intracellular domain of IL7Ra, intracellular domain of IL9Ra, and intracellular domain of IL21Ra.

In some embodiments, wherein said X is selected from anti-CD19 scFv, anti-CD20 scFv, anti-EGFR scFv, anti-HER2 scFv, anti-EGFRVIII scFv, anti-PSMA scFv, anti-BCMA scFv, anti-CD22 scFv, anti-CD30 scFv and anti-CLDN18.2 scFv;

and said Y is an intracellular domain of 4-1BB, said M is an intracellular domain of IL7Ra or intracellular domain of IL21Ra.

In some embodiments, said X is anti-CD20 scFv, and said Y is an intracellular domain of 4-1BB, said M is one selected from intracellular domain of IL2Rb, intracellular domain of IL4Ra, intracellular domain of IL7Ra, intracellular domain of IL9Ra, and intracellular domain of IL21Ra.

In an embodiment, said X is anti-CD20 scFv, and said Y is an intracellular domain of 4-1BB, said M is intracellular domain of IL7Ra.

In some embodiments, said X is anti-CD19 scFv, and said Y is an intracellular domain of 4-1BB, said M is one selected from intracellular domain of IL2Rb, intracellular domain of IL4Ra, intracellular domain of IL7Ra, intracellular domain of IL9Ra, and intracellular domain of IL21Ra.

In an embodiment, said X is anti-CD19 scFv, and said Y is an intracellular domain of 4-1BB, said M is intracellular domain of IL7Ra.

In some embodiments, said X is anti-CLDN18.2 scFv, and said Y is an intracellular domain of 4-1BB, said M is one selected from intracellular domain of IL2Rb, intracellular domain of IL4Ra, intracellular domain of IL7Ra, intracellular domain of IL9Ra, and intracellular domain of IL21Ra.

In an embodiment, said X is anti-CLDN18.2 scFv, and said Y is an intracellular domain of 4-1BB, said M is intracellular domain of IL21Ra.

In some embodiments, said X is anti-EGFR scFv, and said Y is an intracellular domain of 4-1BB, said M is one selected from intracellular domain of IL2Rb, intracellular domain of IL4Ra, intracellular domain of IL7Ra, intracellular domain of IL9Ra, and intracellular domain of IL21Ra.

In an embodiment, said X is anti-EGFR scFv, and said Y is an intracellular domain of 4-1BB, said M is intracellular domain of IL21Ra.

In some embodiments, said chimeric antigen receptor further comprises an extracellular hinge region and a transmembrane domain, said transmembrane domain is selected from CD8a, CD28, CD137 and CD3; said extracellular hinge region is selected from CD8a or IgG.

In some embodiments, said extracellular hinge region and transmembrane domain are derived from the extracellular hinge region and transmembrane domain of CD8a,

In some embodiments, the sequence of said extracellular hinge region and transmembrane domain is as set forth in SEQ ID No: 5.

In some embodiments, said chimeric antigen receptor has a structure of X-H-TM-Y-CD3zeta-M-N;

wherein, X comprises a tumor-targeting antibody or a ligand or receptor capable of specifically binding to a tumor;

H is an extracellular hinge region, said extracellular hinge region is selected from CD8a or IgG;

TM is a transmembrane domain, said transmembrane domain is selected from CD8a, CD28, CD137 and CD3;

Y is an intracellular domain of a costimulatory receptor, said costimulatory receptor is selected from ICOS, CD28, CD27, HVEM, LIGHT, CD40L, 4-1BB, OX40, DR3, GITR, CD30, TIM1, SLAM, CD2, CD226;

M is an intracellular domain of a gamma chain family cytokine receptor, said cytokine receptor is selected from IL2Ra, IL2Rb, IL4Ra, IL7Ra, IL9Ra, IL15Ra, IL21Ra; and

N is an intracellular domain of IL2Rg.

In some embodiments, said chimeric antigen receptor has a structure of X-H-TM-Y-CD3zeta-M-N;

wherein, X comprises a tumor-targeting antibody or a ligand or receptor capable of specifically binding to a tumor;

H is an extracellular hinge region of CD8a;

TM is a transmembrane domain of CD8a;

Y is an intracellular domain of 4-1BB;

M is an intracellular domain of a gamma chain family cytokine receptor, said cytokine receptor is selected from IL2Ra, IL2Rb, IL4Ra, IL7Ra, IL9Ra, IL15Ra, IL21Ra; and

N is an intracellular domain of IL2Rg.

In some embodiments, said chimeric antigen receptor has a structure of X-H-TM-Y-CD3zeta-M-N;

wherein, X is selected from anti-CD19 scFv, anti-CD20 scFv, EGFR scFv, HER2 scFv, EGFRVIII scFv, anti-PSMA scFv, anti-BCMA scFv, anti-CD22 scFv, anti-CD30 scFv and anti-CLDN18.2 scFv;

H is an extracellular hinge region of CD8a;

TM is a transmembrane domain of CD8a;

Y is an intracellular domain of 4-1BB;

M is an intracellular domain of a gamma chain family cytokine receptor, said cytokine receptor is selected from IL2Ra, IL2Rb, IL4Ra, IL7Ra, IL9Ra, IL15Ra, IL21Ra; and

N is an intracellular domain of IL2Rg.

In some embodiments, said chimeric antigen receptor has a structure of X-H-TM-Y-CD3zeta-M-N;

wherein, X is selected from anti-CD19 scFv, anti-CD20 scFv, EGFR scFv, HER2 scFv, EGFRVIII scFv, anti-PSMA scFv, anti-BCMA scFv, anti-CD22 scFv, anti-CD30 scFv and anti-CLDN18.2 scFv;

H is an extracellular hinge region of CD8a;

TM is a transmembrane domain of CD8a;

Y is an intracellular domain of 4-1BB;

M is an intracellular domain of IL7Ra or IL21Ra; and

N is an intracellular domain of IL2Rg.

In some embodiments, the sequence of said H-TM-Y-CD3zeta-M-N is as set forth in any one of SEQ ID No: 23-27.

For example, in an embodiment, said chimeric antigen receptor has a structure of X-H-TM-Y-CD3zeta-M-N;

wherein, X is selected from anti-CD19 scFv, anti-CD20 scFv, EGFR scFv, HER2 scFv, EGFRVIII scFv, anti-PSMA scFv, anti-BCMA scFv, anti-CD22 scFv, anti-CD30 scFv and anti-CLDN18.2 scFv; and the sequence of said H-TM-Y-CD3zeta-M-N is as set forth in any one of SEQ ID No: 23-27.

In some embodiments, said chimeric antigen receptor has a structure of X-H-TM-Y-CD3 zeta-M-N;

wherein, X is selected from anti-CD19 scFv, anti-CD20 scFv, anti-EGFR scFv, anti-HER2 scFv, anti-EGFRVIII scFv, anti-PSMA scFv, anti-BCMA scFv, anti-CD22 scFv, anti-CD30 scFv and anti-CLDN18.2 scFv;

H is an extracellular hinge region, said extracellular hinge region is selected from CD8a or IgG;

TM is a transmembrane domain, said transmembrane domain is selected from CD8a, CD28, CD137 and CD3;

Y is an intracellular domain of a costimulatory receptor, said costimulatory receptor is selected from ICOS, CD28, CD27, HVEM, LIGHT, CD40L, 4-1BB, OX40, DR3, GITR, CD30, TIM1, SLAM, CD2, CD226;

M is an intracellular domain of a gamma chain family cytokine receptor, said cytokine receptor is selected from IL2Ra, IL2Rb, IL4Ra, IL7Ra, IL9Ra, IL15Ra, IL21Ra; and

N is an intracellular domain of IL2Rg.

In some embodiments, said chimeric antigen receptor has a structure of X-H-TM-Y-CD3 zeta-M-N;

wherein, X is selected from anti-CD19 scFv, anti-CD20 scFv, anti-EGFR scFv, anti-HER2 scFv, anti-EGFRVIII scFv, anti-PSMA scFv, anti-BCMA scFv, anti-CD22 scFv, anti-CD30 scFv and anti-CLDN18.2 scFv;

H is an extracellular hinge region of CD8a;

TM is a transmembrane domain of CD8;

Y is an intracellular domain of a 4-1BB;

M is an intracellular domain of a gamma chain family cytokine receptor, said cytokine receptor is selected from IL2Ra, IL2Rb, IL4Ra, IL7Ra, IL9Ra, IL15Ra, IL21Ra; and

N is an intracellular domain of IL2Rg.

In some embodiments, said X is anti-CD20 scFv. In an embodiment, the sequence of said anti-CD20 scFv is set forth in SEQ ID NO: 1. Further, the sequence of said anti-CD20 scFv(X)-H-TM-Y-CD3zeta is set forth in SEQ ID NO: 15.

For example, said chimeric antigen receptor may have a structure of X-H-TM-Y-CD3zeta-M-N, wherein said X is anti-CD20 scFv, the sequence of said anti-CD20 scFv(X)-H-TM-Y-CD3zeta is set forth in SEQ ID NO: 15; M is an intracellular domain of a gamma chain family cytokine receptor, said cytokine receptor is selected from IL2Ra, IL2Rb, IL4Ra, IL7Ra, IL9Ra, IL15Ra, IL21Ra; and N is an intracellular domain of IL2Rg.

In some embodiments, said X is anti-CD19 scFv. In an embodiment, the sequence of said anti-CD19 scFv is set forth in SEQ ID NO: 2. Further, the sequence of said anti-CD19 scFv(X)-H-TM-Y-CD3zeta is set forth in SEQ ID NO: 17.

For example, said chimeric antigen receptor may have a structure of X-H-TM-Y-CD3zeta-M-N, wherein said X is anti-CD19 scFv, the sequence of said anti-CD19 scFv(X)-H-TM-Y-CD3zeta is set forth in SEQ ID NO: 17; M is an intracellular domain of a gamma chain family cytokine receptor, said cytokine receptor is selected from IL2Ra, IL2Rb, IL4Ra, IL7Ra, IL9Ra, IL15Ra, IL21Ra; and N is an intracellular domain of IL2Rg.

In some embodiments, said X is anti-CLDN18.2 scFv. In an embodiment, the sequence of said anti-CLDN18.2 scFv is set forth in SEQ ID NO: 3. Further, the sequence of said anti-CLDN18.2 scFv(X)-H-TM-Y-CD3zeta is set forth in SEQ ID NO: 19.

For example, said chimeric antigen receptor may have a structure of X-H-TM-Y-CD3zeta-M-N, wherein said X is anti-CLDN18.2 scFv, the sequence of said anti-CLDN18.2 scFv(X)-H-TM-Y-CD3zeta is set forth in SEQ ID NO: 19; M is an intracellular domain of a gamma chain family cytokine receptor, said cytokine receptor is selected from IL2Ra, IL2Rb, IL4Ra, IL7Ra, IL9Ra, IL15Ra, IL21Ra; and N is an intracellular domain of IL2Rg.

In some embodiments, said X is anti-EGFR scFv. In an embodiment, the sequence of said anti-EGFR scFv is set forth in SEQ ID NO: 4. Further, the sequence of said anti-EGFR scFv(X)-H-TM-Y-CD3zeta is set forth in SEQ ID NO: 21.

For example, said chimeric antigen receptor may have a structure of X-H-TM-Y-CD3zeta-M-N, wherein said X is anti-EGFR scFv, the sequence of said anti-EGFR scFv(X)-H-TM-Y-CD3zeta is set forth in SEQ ID NO: 21; M is an intracellular domain of a gamma chain family cytokine receptor, said cytokine receptor is selected from IL2Ra, IL2Rb, IL4Ra, IL7Ra, IL9Ra, IL15Ra, IL21Ra; and N is an intracellular domain of IL2Rg.

In some embodiments, said chimeric antigen receptor has a structure of X-H-TM-Y-CD3 zeta-M-N;

wherein, X is anti-CD20 scFv;

H is an extracellular hinge region of CD8a;

TM is a transmembrane domain of CD8;

Y is an intracellular domain of a 4-1BB;

M is an intracellular domain of IL7Ra; and

N is an intracellular domain of IL2Rg.

In an embodiment, the sequence of said anti-CD20 scFv is set forth in SEQ ID NO: 1. Further, the sequence of said anti-CD20 scFv(X)-H-TM-Y-CD3zeta is set forth in SEQ ID NO: 15. Further, the sequence of said anti-CD20 scFv(X)-H-TM-Y-CD3zeta-M-N is set forth in SEQ ID NO: 16.

In some embodiments, said chimeric antigen receptor has a structure of X-H-TM-Y-CD3 zeta-M-N;

wherein, X is anti-CD19 scFv;

H is an extracellular hinge region of CD8a;

TM is a transmembrane domain of CD8;

Y is an intracellular domain of a 4-1BB;

M is an intracellular domain of IL7Ra; and

N is an intracellular domain of IL2Rg.

In an embodiment, the sequence of said anti-CD19 scFv is set forth in SEQ ID NO: 2. Further, the sequence of said anti-CD19 scFv(X)-H-TM-Y-CD3zeta is set forth in SEQ ID NO: 17. Further, the sequence of said anti-CD19 scFv(X)-H-TM-Y-CD3zeta-M-N is set forth in SEQ ID NO: 18.

In some embodiments, said chimeric antigen receptor has a structure of X-H-TM-Y-CD3zeta-M-N;

wherein, X is anti-CLDN18.2 scFv;

H is an extracellular hinge region of CD8a;

TM is a transmembrane domain of CD8;

Y is an intracellular domain of a 4-1BB;

M is an intracellular domain of IL21Ra; and

N is an intracellular domain of IL2Rg.

In an embodiment, the sequence of said anti-CLDN18.2 scFv is set forth in SEQ ID NO: 3. Further, the sequence of said anti-CLDN18.2 scFv(X)-H-TM-Y-CD3zeta is set forth in SEQ ID NO: 19. Further, the sequence of said anti-CLDN18.2 scFv(X)-H-TM-Y-CD3zeta-M-N is set forth in SEQ ID NO: 20.

In some embodiments, said chimeric antigen receptor has a structure of X-H-TM-Y-CD3zeta-M-N;

wherein, X is anti-EGFR scFv;

H is an extracellular hinge region of CD8a;

TM is a transmembrane domain of CD8;

Y is an intracellular domain of a 4-1BB;

M is an intracellular domain of IL21Ra; and

N is an intracellular domain of IL2Rg.

In an embodiment, the sequence of said anti-EGFR scFv is set forth in SEQ ID NO: 4. Further, the sequence of said anti-EGFR scFv(X)-H-TM-Y-CD3zeta is set forth in SEQ ID NO: 21. Further, the sequence of said anti-EGFR scFv(X)-H-TM-Y-CD3zeta-M-N is set forth in SEQ ID NO: 22.

In some embodiments, said chimeric antigen receptor further comprises a signal peptide.

In some embodiments, said signal peptide of a protein selected from the group consisting of CD8, CD28, GM-CSF, CD4, CD137, and a combination thereof.

In some embodiments, said signal peptide derived from CD8.

In some embodiments, the sequence of said signal peptide is set forth in SEQ ID NO: 14.

In some embodiments, said chimeric antigen receptor has a structure of L-X-H-TM-Y-CD3zeta-M-N;

wherein, X is selected from anti-CD19 scFv, anti-CD20 scFv, anti-EGFR scFv, anti-HER2 scFv, anti-EGFRVIII scFv, anti-PSMA scFv, anti-BCMA scFv, anti-CD22 scFv, anti-CD30 scFv and anti-CLDN18.2 scFv;

L is a signal peptide derived from CD8;

H is an extracellular hinge region, said extracellular hinge region is selected from CD8a or IgG;

TM is a transmembrane domain, said transmembrane domain is selected from CD8a, CD28, CD137 and CD3;

Y is an intracellular domain of a costimulatory receptor, said costimulatory receptor is selected from ICOS, CD28, CD27, HVEM, LIGHT, CD40L, 4-1BB, OX40, DR3, GITR, CD30, TIM1, SLAM, CD2, CD226;

M is an intracellular domain of a gamma chain family cytokine receptor, said cytokine receptor is selected from IL2Ra, IL2Rb, IL4Ra, IL7Ra, IL9Ra, IL15Ra, IL21Ra; and

N is an intracellular domain of IL2Rg.

The instant invention also provides an isolated nucleic acid molecule comprising a polynucleotide encoding a chimeric antigen receptor comprising the third signal receptor as described in the present application.

The instant invention also provides an expression vector, comprising the polynucleotide encoding the chimeric antigen receptor comprising the third signal receptor as described in the present application.

In some embodiments, the vector comprises DNA and RNA.

In some embodiments, the vector is selected from the group consisting of plasmid, virus vector, transposon, and a combination thereof.

In some embodiments, the vector comprises a DNA virus and a retrovirus vector.

In some embodiments, the vector is selected from the group consisting of a lentiviral vector, an adenovirus vector, an adeno-associated virus vector, and a combination thereof.

In some embodiments, the vector is a lentiviral vector.

In another aspect of the invention, it provides a host cell, comprising the vector of the third aspect of the invention or having the exogenous nucleic acid molecule of the second aspect of the invention integrated into its genome or expressing the chimeric antigen receptor of the first aspect of the invention.

In some embodiments, the cell is an isolated cell.

In some embodiments, the cell is a genetically engineered cell.

In some embodiments, the cell is a mammalian cell.

In some embodiments, the cell is a CAR-T cell and/or a CAR-NK cell.

The instant invention also provides a chimeric antigen receptor-T (CAR-T) cell comprising an expression vector encoding and expressing the CAR as described in the present application.

The instant invention also provides a method of preventing or treating a tumor, comprising administrating said CAR-T cell as described in the present application to a subject in need thereof.

Wherein, each aforesaid sequence is specifically as follows:

| ID | NAME | SEQ |
|---|---|---|
| 1 | anti-CD20 scFv | QIVLSQSPAILSASPGEKVTMTCRASSSVSYIHWFQQKPGSSPKPWI |
| | | YATSNLASGVPVRFSGSGSGTSYSLTISRVEAEDAATYYCQQWTS |
| | | NPPTFGGGTKLEIKGGGGSGGGGSGGGGSQVQLQQPGAELVKPG |
| | | ASVKMSCKASGYTFTSYNMHWVKQTPGRGLEWIGAIYPGNGDTS |
| | | YNQKFKGKATLTADKSSSTAYMQLSSLTSEDSAVYYCARSTYYG |
| | | GDWYFNVWGAGTTVTVS |
| 2 | anti-CD19 scFv | DIQMTQTTSSLSASLGDRVTISCRASQDISKYLNWYQQKPDGTVK |
| | | LLIYHTSRLHSGVPSRFSGSGSGTDYSLTISNLEQEDIATYFCQQGN |
| | | TLPYTFGGGTKLEITGGGGSGGGGSGGGGSEVKLQESGPGLVAPS |
| | | QSLSVTCTVSGVSLPDYGVSWIRQPPRKGLEWLGVIWGSETTYYN |
| | | SALKSRLTIIKDNSKSQVFLKMNSLQTDDTAIYYCAKHYYYGGSY |
| | | AMDYWGQGTSVTVSS |
| 3 | anti-CLDN18.2 | DIVMTQSPDSLAVSLGERATISCKSSQSLLNSGNQKNYLTWYQQK |
| | scFv | PGQPPKLLIYWASTRESGVPDRFSGSGSGTDFTLTISSLQAEDVAV |
| | | YYCQNDYFYPFTFGQGTKLEIKRTVGGGGSGGGGSGGGGSQVQL |
| | | VQSGAEVKKPGSSVKVSCKASGYAFSNYLIEWVKQAPGQGLEWI |
| | | GLINPGSGGTNYNEKFKGKATITADKSTSTAYMELSSLRSEDTAV |
| | | YYCARVYYGNSFAYWGQGTLVTVSS |
| 4 | anti-EGFR scFv | QVQLKQSGPGLVQPSQSLSITCTVSGFSLTNYGVHWVRQSPGKGL |
| | | EWLGVIWSGGNTDYNTPFTSRLSINKDNSKSQVFFKMNSLQSNDT |
| | | AIYYCARALTYYDYEFAYWGQGTLVTVSAGGGGSGGGGSGGGG |
| | | SDILLTQSPVILSVSPGERVSFSCRASQSIGTNIHWYQQRTNGSPRL |
| | | LIKYASESISGIPSRFSGSGSGTDFTLSINSVESEDIADYYCQQNNN |
| | | WPTTFGAGTKLELK |
| 5 | CD8a | TTTPAPRPPTPAPTIASQPLSLRPEACRPAAGGAVHTRGLDFACDIY |
| | extracellular | IWAPLAGTCGVLLLSLVITLYC |
| | hinge region and | |
| | transmembrane | |
| | domain | |
| 6 | intracellular | KRGRKKLLYIFKQPFMRPVQTTQEEDGCSCRFPEEEEGGCEL |
| | domain of 4- | |
| | 1BB | |
| 7 | intracellular | RVKFSRSADAPAYQQGQNQLYNELNLGRREEYDVLDKRRGRDPE |
| | domain of | MGGKPRRKNPQEGLYNELQKDKMAEAYSEIGMKGERRRGKGHD |
| | CD3zeta | GLYQGLSTATKDTYDALHMQALPPR |
| 8 | intracellular | KKRIKPIVWPSLPDHKKTLEHLCKKPRKNLNVSFNPESFLDCQIHR |
| | domain of | VDDIQARDEVEGFLQDTFPQQLEESEKQRLGGDVQSPNCPSEDVV |
| | IL7Ra | ITPESFGRDSSLTCLAGNVSACDAPILSSSRSLDCRESGKNGPHVYQ |
| | | DLLLSLGTTNSTLPPPFSLQSGILTLNPVAQGQPILTSLGSNQEEAY |
| | | VTMSSFYQNQ |
| 9 | intracellular | NCRNTGPWLKKVLKCNTPDPSKFFSQLSSEHGGDVQKWLSSPFPS |
| | domain of | SSFSPGGLAPEISPLEVLERDKVTQLLLQQDKVPEPASLSSNHSLTS |
| | IL2Rb | CFTNQGYFFFHLPDALEIEACQVYFTYDPYSEEDPDEGVAGAPTGS |
| | | SPQPLQPLSGEDDAYCTFPSRDDLLLFSPSLLGGPSPPSTAPGGSGA |
| | | GEERMPPSLQERVPRDWDPQPLGPPTPGVPDLVDFQPPPELVLRE |
| | | AGEEVPDAGPREGVSFPWSRPPGQGEFRALNARLPLNTDAYLSLQ |
| | | ELQGQDPTHLV |
| 10 | intracellular | KIKKEWWDQIPNPARSRLVAIIIQDAQGSQWEKRSRGQEPAKCPH |
| | domain of | WKNCLTKLLPCFLEHNMKRDEDPHKAAKEMPFQGSGKSAWCPV |
| | IL4Ra | EISKTVLWPESISVVRCVELFEAPVECEEEEEVEEEKGSFCASPESS |
| | | RDDFQEGREGIVARLTESLFLDLLGEENGGFCQQDMGESCLLPPS |
| | | GSTSAHMPWDEFPSAGPKEAPPWGKEQPLHLEPSPPASPTQSPDN |
| | | LTCTETPLVIAGNPAYRSFSNSLSQSPCPRELGPDPLLARHLEEVEP |
| | | EMPCVPQLSEPTTVPQPEPETWEQILRRNVLQHGAAAAPVSAPTS |
| | | GYQEFVHAVEQGGTQASAVVGLGPPGEAGYKAFSSLLASSAVSPE |
| | | KCGFGASSGEEGYKPFQDLIPGCPGDPAPVPVPLFTFGLDREPPRSP |
| | | QSSHLPSSSPEHLGLEPGEKVEDMPKPPLPQEQATDPLVDSLGSGI |
| | | VYSALTCHLCGHLKQCHGQEDGGQTPVMASPCCGCCCGDRSSPP |
| | | TTPLRAPDPSPGGVPLEASLCPASLAPSGISEKSKSSSSFHPAPGNA |
| | | QSSSQTPKIVNFVSVGPTYMRVS |
| 11 | intracellular | KLSPRVKRIFYQNVPSPAMFFQPLYSVHNGNFQTWMGAHGAGVL |
| | domain of | LSQDCAGTPQGALEPCVQEATALLTCGPARPWKSVALEEEQEGPG |
| | IL9Ra | TRLPGNLSSEDVLPAGCTEWRVQTLAYLPQEDWAPTSLTRPAPPD |
| | | SEGSRSSSSSSSSNNNNYCALGCYGGWHLSALPGNTQSSGPIPALA |
| | | CGLSCDHQGLETQQGVAWVLAGHCQRPGLHEDLQGMLLPSVLS |
| | | KARSWTF |
| 12 | intracellular | SLKTHPLWRLWKKIWAVPSPERFFMPLYKGCSGDFKKWVGAPFT |
| | domain of | GSSLELGPWSPEVPSTLEVYSCHPPRSPAKRLQLTELQEPAELVES |
| | IL21Ra | DGVPKPSFWPTAQNSGGSAYSEERDRPYGLVSIDTVTVLDAEGPC |
| | | TWPCSCEDDGYPALDLDAGLEPSPGLEDPLLDAGTTVLSCGCVSA |
| | | GSPGLGGPLGSLLDRLKPPLADGEDWAGGLPWGGRSPGGVSESE |
| | | AGSPLAGLDMDTFDSGFVGSDCSSPVECDFTSPGDEGPPRSYLRQ |
| | | WVVIPPPLSSPGPQAS |
| 13 | intracellular | ERTMPRIPTLKNLEDLVTEYHGNFSAWSGVSKGLAESLQPDYSER |
| | domain of | LCLVSEIPPKGGALGEGPGASPCNQHSPYWAPPCYTLKPET |
| | IL2Rg | |
| 14 | signal peptide | METDTLLLWVLLLWVPGSTGTG |
| 15 | anti-CD20 scFv | QIVLSQSPAILSASPGEKVTMTCRASSSVSYIHWFQQKPGSSPKPWI |
| | (X)-H-TM-Y- | YATSNLASGVPVRFSGSGSGTSYSLTISRVEAEDAATYYCQQWTS |
| | CD3zeta | NPPTFGGGTKLEIKGGGGSGGGGSGGGGSQVQLQQPGAELVKPG |
| | | ASVKMSCKASGYTFTSYNMHWVKQTPGRGLEWIGAIYPGNGDTS |
| | | YNQKFKGKATLTADKSSSTAYMQLSSLTSEDSAVYYCARSTYYG |
| | | GDWYFNVWGAGTTVTVSAAAATTTPAPRPPTPAPTIASQPLSLRP |
| | | EACRPAAGGAVHTRGLDFACDIYIWAPLAGTCGVLLLSLVITLYC |
| | | KRGRKKLLYIFKQPFMRPVQTTQEEDGCSCRFPEEEEGGCELRVK |
| | | FSRSADAPAYQQGQNQLYNELNLGRREEYDVLDKRRGRDPEMG |
| | | GKPRRKNPQEGLYNELQKDKMAEAYSEIGMKGERRRGKGHDGL |
| | | YQGLSTATKDTYDALHMQALPPR |
| 16 | anti-CD20 scFv | QIVLSQSPAILSASPGEKVTMTCRASSSVSYIHWFQQKPGSSPKPWI |
| | (X)-H-TM-Y- | YATSNLASGVPVRFSGSGSGTSYSLTISRVEAEDAATYYCQQWTS |
| | CD3zeta-M-N | NPPTFGGGTKLEIKGGGGSGGGGSGGGGSQVQLQQPGAELVKPG |
| | | ASVKMSCKASGYTFTSYNMHWVKQTPGRGLEWIGAIYPGNGDTS |
| | | YNQKFKGKATLTADKSSSTAYMQLSSLTSEDSAVYYCARSTYYG |
| | | GDWYFNVWGAGTTVTVSAAAATTTPAPRPPTPAPTIASQPLSLRP |
| | | EACRPAAGGAVHTRGLDFACDIYIWAPLAGTCGVLLLSLVITLYC |
| | | KRGRKKLLYIFKQPFMRPVQTTQEEDGCSCRFPEEEEGGCELRVK |
| | | FSRSADAPAYQQGQNQLYNELNLGRREEYDVLDKRRGRDPEMG |
| | | GKPRRKNPQEGLYNELQKDKMAEAYSEIGMKGERRRGKGHDGL |
| | | YQGLSTATKDTYDALHMQALPPRKKRIKPIVWPSLPDHKKTLEHL |
| | | CKKPRKNLNVSFNPESFLDCQIHRVDDIQARDEVEGFLQDTFPQQL |
| | | EESEKQRLGGDVQSPNCPSEDVVITPESFGRDSSLTCLAGNVSACD |
| | | APILSSSRSLDCRESGKNGPHVYQDLLLSLGTTNSTLPPPFSLQSGIL |
| | | TLNPVAQGQPILTSLGSNQEEAYVTMSSFYQNQERTMPRIPTLKNL |
| | | EDLVTEYHGNFSAWSGVSKGLAESLQPDYSERLCLVSEIPPKGGA |
| | | LGEGPGASPCNQHSPYWAPPCYTLKPET |
| 17 | anti-CD19 scFv | DIQMTQTTSSLSASLGDRVTISCRASQDISKYLNWYQQKPDGTVK |
| | (X)-H-TM-Y- | LLIYHTSRLHSGVPSRFSGSGSGTDYSLTISNLEQEDIATYFCQQGN |
| | CD3zeta | TLPYTFGGGTKLEITGGGGSGGGGSGGGGSEVKLQESGPGLVAPS |
| | | QSLSVTCTVSGVSLPDYGVSWIRQPPRKGLEWLGVIWGSETTYYN |
| | | SALKSRLTIIKDNSKSQVFLKMNSLQTDDTAIYYCAKHYYYGGSY |
| | | AMDYWGQGTSVTVSSAAATTTPAPRPPTPAPTIASQPLSLRPEACR |
| | | PAAGGAVHTRGLDFACDIYIWAPLAGTCGVLLLSLVITLYCKRGR |
| | | KKLLYIFKQPFMRPVQTTQEEDGCSCRFPEEEEGGCELRVKFSRSA |
| | | DAPAYQQGQNQLYNELNLGRREEYDVLDKRRGRDPEMGGKPRR |
| | | KNPQEGLYNELQKDKMAEAYSEIGMKGERRRGKGHDGLYQGLS |
| | | TATKDTYDALHMQALPPR |
| 18 | anti-CD19 scFv | DIQMTQTTSSLSASLGDRVTISCRASQDISKYLNWYQQKPDGTVK |
| | (X)-H-TM-Y- | LLIYHTSRLHSGVPSRFSGSGSGTDYSLTISNLEQEDIATYFCQQGN |
| | CD3zeta-M-N | TLPYTFGGGTKLEITGGGGSGGGGSGGGGSEVKLQESGPGLVAPS |
| | | QSLSVTCTVSGVSLPDYGVSWIRQPPRKGLEWLGVIWGSETTYYN |
| | | SALKSRLTIIKDNSKSQVFLKMNSLQTDDTAIYYCAKHYYYGGSY |
| | | AMDYWGQGTSVTVSSAAATTTPAPRPPTPAPTIASQPLSLRPEACR |
| | | PAAGGAVHTRGLDFACDIYIWAPLAGTCGVLLLSLVITLYCKRGR |
| | | KKLLYIFKQPFMRPVQTTQEEDGCSCRFPEEEEGGCELRVKFSRSA |
| | | DAPAYQQGQNQLYNELNLGRREEYDVLDKRRGRDPEMGGKPRR |
| | | KNPQEGLYNELQKDKMAEAYSEIGMKGERRRGKGHDGLYQGLS |
| | | TATKDTYDALHMQALPPRKKRIKPIVWPSLPDHKKTLEHLCKKPR |
| | | KNLNVSFNPESFLDCQIHRVDDIQARDEVEGFLQDTFPQQLEESEK |
| | | QRLGGDVQSPNCPSEDVVITPESFGRDSSLTCLAGNVSACDAPILS |
| | | SSRSLDCRESGKNGPHVYQDLLLSLGTTNSTLPPPFSLQSGILTLNP |
| | | VAQGQPILTSLGSNQEEAYVTMSSFYQNQERTMPRIPTLKNLEDL |
| | | VTEYHGNFSAWSGVSKGLAESLQPDYSERLCLVSEIPPKGGALGE |
| | | GPGASPCNQHSPYWAPPCYTLKPET |
| 19 | anti-CLDN18.2 | DIVMTQSPDSLAVSLGERATISCKSSQSLLNSGNQKNYLTWYQQK |
| | scFv (X)-H-TM- | PGQPPKLLIYWASTRESGVPDRFSGSGSGTDFTLTISSLQAEDVAV |
| | Y-CD3zeta | YYCQNDYFYPFTFGQGTKLEIKRTVGGGGSGGGGSGGGGSQVQL |
| | | VQSGAEVKKPGSSVKVSCKASGYAFSNYLIEWVKQAPGQGLEWI |
| | | GLINPGSGGTNYNEKFKGKATITADKSTSTAYMELSSLRSEDTAV |
| | | YYCARVYYGNSFAYWGQGTLVTVSSAAATTTPAPRPPTPAPTIAS |
| | | QPLSLRPEACRPAAGGAVHTRGLDFACDIYIWAPLAGTCGVLLLS |
| | | LVITLYCKRGRKKLLYIFKQPFMRPVQTTQEEDGCSCRFPEEEEGG |
| | | CELRVKFSRSADAPAYQQGQNQLYNELNLGRREEYDVLDKRRGR |
| | | DPEMGGKPRRKNPQEGLYNELQKDKMAEAYSEIGMKGERRRGK |
| | | GHDGLYQGLSTATKDTYDALHMQALPPR |
| 20 | anti-CLDN18.2 | DIVMTQSPDSLAVSLGERATISCKSSQSLLNSGNQKNYLTWYQQK |
| | scFv (X)-H-TM- | PGQPPKLLIYWASTRESGVPDRFSGSGSGTDFTLTISSLQAEDVAV |
| | Y-CD3zeta-M- | YYCQNDYFYPFTFGQGTKLEIKRTVGGGGSGGGGSGGGGSQVQL |
| | N | VQSGAEVKKPGSSVKVSCKASGYAFSNYLIEWVKQAPGQGLEWI |
| | | GLINPGSGGTNYNEKFKGKATITADKSTSTAYMELSSLRSEDTAV |
| | | YYCARVYYGNSFAYWGQGTLVTVSSAAATTTPAPRPPTPAPTIAS |
| | | QPLSLRPEACRPAAGGAVHTRGLDFACDIYIWAPLAGTCGVLLLS |
| | | LVITLYCKRGRKKLLYIFKQPFMRPVQTTQEEDGCSCRFPEEEEGG |
| | | CELRVKFSRSADAPAYQQGQNQLYNELNLGRREEYDVLDKRRGR |
| | | DPEMGGKPRRKNPQEGLYNELQKDKMAEAYSEIGMKGERRRGK |
| | | GHDGLYQGLSTATKDTYDALHMQALPPRSLKTHPLWRLWKKIW |
| | | AVPSPERFFMPLYKGCSGDFKKWVGAPFTGSSLELGPWSPEVPST |
| | | LEVYSCHPPRSPAKRLQLTELQEPAELVESDGVPKPSFWPTAQNSG |
| | | GSAYSEERDRPYGLVSIDTVTVLDAEGPCTWPCSCEDDGYPALDL |
| | | DAGLEPSPGLEDPLLDAGTTVLSCGCVSAGSPGLGGPLGSLLDRL |
| | | KPPLADGEDWAGGLPWGGRSPGGVSESEAGSPLAGLDMDTFDSG |
| | | FVGSDCSSPVECDFTSPGDEGPPRSYLRQWVVIPPPLSSPGPQASER |
| | | TMPRIPTLKNLEDLVTEYHGNFSAWSGVSKGLAESLQPDYSERLC |
| | | LVSEIPPKGGALGEGPGASPCNQHSPYWAPPCYTLKPET |
| 21 | anti-EGFR scFv | QVQLKQSGPGLVQPSQSLSITCTVSGFSLTNYGVHWVRQSPGKGL |
| | (X)-H-TM-Y- | EWLGVIWSGGNTDYNTPFTSRLSINKDNSKSQVFFKMNSLQSNDT |
| | CD3zeta | AIYYCARALTYYDYEFAYWGQGTLVTVSAGGGGSGGGGSGGGG |
| | | SDILLTQSPVILSVSPGERVSFSCRASQSIGTNIHWYQQRTNGSPRL |
| | | LIKYASESISGIPSRFSGSGSGTDFTLSINSVESEDIADYYCQQNNN |
| | | WPTTFGAGTKLELKAAATTTPAPRPPTPAPTIASQPLSLRPEACRP |
| | | AAGGAVHTRGLDFACDIYIWAPLAGTCGVLLLSLVITLYCKRGRK |
| | | KLLYIFKQPFMRPVQTTQEEDGCSCRFPEEEEGGCELRVKFSRSAD |
| | | APAYQQGQNQLYNELNLGRREEYDVLDKRRGRDPEMGGKPRRK |
| | | NPQEGLYNELQKDKMAEAYSEIGMKGERRRGKGHDGLYQGLST |
| | | ATKDTYDALHMQALPPR |
| 22 | anti-EGFR scFv | QVQLKQSGPGLVQPSQSLSITCTVSGFSLTNYGVHWVRQSPGKGL |
| | (X)-H-TM-Y- | EWLGVIWSGGNTDYNTPFTSRLSINKDNSKSQVFFKMNSLQSNDT |
| | CD3zeta-M-N | AIYYCARALTYYDYEFAYWGQGTLVTVSAGGGGSGGGGSGGGG |
| | | SDILLTQSPVILSVSPGERVSFSCRASQSIGTNIHWYQQRTNGSPRL |
| | | LIKYASESISGIPSRFSGSGSGTDFTLSINSVESEDIADYYCQQNNN |
| | | WPTTFGAGTKLELKAAATTTPAPRPPTPAPTIASQPLSLRPEACRP |
| | | AAGGAVHTRGLDFACDIYIWAPLAGTCGVLLLSLVITLYCKRGRK |
| | | KLLYIFKQPFMRPVQTTQEEDGCSCRFPEEEEGGCELRVKFSRSAD |
| | | APAYQQGQNQLYNELNLGRREEYDVLDKRRGRDPEMGGKPRRK |
| | | NPQEGLYNELQKDKMAEAYSEIGMKGERRRGKGHDGLYQGLST |
| | | ATKDTYDALHMQALPPRSLKTHPLWRLWKKIWAVPSPERFFMPL |
| | | YKGCSGDFKKWVGAPFTGSSLELGPWSPEVPSTLEVYSCHPPRSP |
| | | AKRLQLTELQEPAELVESDGVPKPSFWPTAQNSGGSAYSEERDRP |
| | | YGLVSIDTVTVLDAEGPCTWPCSCEDDGYPALDLDAGLEPSPGLE |
| | | DPLLDAGTTVLSCGCVSAGSPGLGGPLGSLLDRLKPPLADGEDWA |
| | | GGLPWGGRSPGGVSESEAGSPLAGLDMDTFDSGFVGSDCSSPVEC |
| | | DFTSPGDEGPPRSYLRQWVVIPPPLSSPGPQASERTMPRIPTLKNLE |
| | | DLVTEYHGNFSAWSGVSKGLAESLQPDYSERLCLVSEIPPKGGAL |
| | | GEGPGASPCNQHSPYWAPPCYTLKPET |
| 23 | CD8(hinge/TM)- | TTTPAPRPPTPAPTIASQPLSLRPEACRPAAGGAVHTRGLDFACDIY |
| | 4-1BB- | IWAPLAGTCGVLLLSLVITLYCKRGRKKLLYIFKQPFMRPVQTTQE |
| | CD3zeta- | EDGCSCRFPEEEEGGCELRVKFSRSADAPAYQQGQNQLYNELNLG |
| | IL7Ra-IL2Rg | RREEYDVLDKRRGRDPEMGGKPRRKNPQEGLYNELQKDKMAEA |
| | | YSEIGMKGERRRGKGHDGLYQGLSTATKDTYDALHMQALPPRK |
| | | KRIKPIVWPSLPDHKKTLEHLCKKPRKNLNVSFNPESFLDCQIHRV |
| | | DDIQARDEVEGFLQDTFPQQLEESEKQRLGGDVQSPNCPSEDVVIT |
| | | PESFGRDSSLTCLAGNVSACDAPILSSSRSLDCRESGKNGPHVYQD |
| | | LLLSLGTTNSTLPPPFSLQSGILTLNPVAQGQPILTSLGSNQEEAYV |
| | | TMSSFYQNQERTMPRIPTLKNLEDLVTEYHGNFSAWSGVSKGLAE |
| | | SLQPDYSERLCLVSEIPPKGGALGEGPGASPCNQHSPYWAPPCYTL |
| | | KPET |
| 24 | CD8(hinge/TM)- | TTTPAPRPPTPAPTIASQPLSLRPEACRPAAGGAVHTRGLDFACDIY |
| | 4-1BB- | IWAPLAGTCGVLLLSLVITLYCKRGRKKLLYIFKQPFMRPVQTTQE |
| | CD3zeta- | EDGCSCRFPEEEEGGCELRVKFSRSADAPAYQQGQNQLYNELNLG |
| | IL21Ra-IL2Rg | RREEYDVLDKRRGRDPEMGGKPRRKNPQEGLYNELQKDKMAEA |
| | | YSEIGMKGERRRGKGHDGLYQGLSTATKDTYDALHMQALPPRSL |
| | | KTHPLWRLWKKIWAVPSPERFFMPLYKGCSGDFKKWVGAPFTGS |
| | | SLELGPWSPEVPSTLEVYSCHPPRSPAKRLQLTELQEPAELVESDG |
| | | VPKPSFWPTAQNSGGSAYSEERDRPYGLVSIDTVTVLDAEGPCTW |
| | | PCSCEDDGYPALDLDAGLEPSPGLEDPLLDAGTTVLSCGCVSAGS |
| | | PGLGGPLGSLLDRLKPPLADGEDWAGGLPWGGRSPGGVSESEAG |
| | | SPLAGLDMDTFDSGFVGSDCSSPVECDFTSPGDEGPPRSYLRQWV |
| | | VIPPPLSSPGPQASERTMPRIPTLKNLEDLVTEYHGNFSAWSGVSK |
| | | GLAESLQPDYSERLCLVSEIPPKGGALGEGPGASPCNQHSPYWAPP |
| | | CYTLKPET |
| 25 | CD8(hinge/TM)- | TTTPAPRPPTPAPTIASQPLSLRPEACRPAAGGAVHTRGLDFACDIY |
| | 4-1BB- | IWAPLAGTCGVLLLSLVITLYCKRGRKKLLYIFKQPFMRPVQTTQE |
| | CD3zeta- | EDGCSCRFPEEEEGGCELRVKFSRSADAPAYQQGQNQLYNELNLG |
| | IL2Rb-IL2Rg | RREEYDVLDKRRGRDPEMGGKPRRKNPQEGLYNELQKDKMAEA |
| | | YSEIGMKGERRRGKGHDGLYQGLSTATKDTYDALHMQALPPRNC |
| | | RNTGPWLKKVLKCNTPDPSKFFSQLSSEHGGDVQKWLSSPFPSSSF |
| | | SPGGLAPEISPLEVLERDKVTQLLLQQDKVPEPASLSSNHSLTSCFT |
| | | NQGYFFFHLPDALEIEACQVYFTYDPYSEEDPDEGVAGAPTGSSPQ |
| | | PLQPLSGEDDAYCTFPSRDDLLLFSPSLLGGPSPPSTAPGGSGAGEE |
| | | RMPPSLQERVPRDWDPQPLGPPTPGVPDLVDFQPPPELVLREAGE |
| | | EVPDAGPREGVSFPWSRPPGQGEFRALNARLPLNTDAYLSLQELQ |
| | | GQDPTHLVERTMPRIPTLKNLEDLVTEYHGNFSAWSGVSKGLAES |
| | | LQPDYSERLCLVSEIPPKGGALGEGPGASPCNQHSPYWAPPCYTL |
| | | KPET |
| 26 | CD8(hinge/TM)- | TTTPAPRPPTPAPTIASQPLSLRPEACRPAAGGAVHTRGLDFACDIY |
| | 4-1BB- | IWAPLAGTCGVLLLSLVITLYCKRGRKKLLYIFKQPFMRPVQTTQE |
| | CD3zeta-IL4Ra- | EDGCSCRFPEEEEGGCELRVKFSRSADAPAYQQGQNQLYNELNLG |
| | IL2Rg | RREEYDVLDKRRGRDPEMGGKPRRKNPQEGLYNELQKDKMAEA |
| | | YSEIGMKGERRRGKGHDGLYQGLSTATKDTYDALHMQALPPRKI |
| | | KKEWWDQIPNPARSRLVAIIIQDAQGSQWEKRSRGQEPAKCPHW |
| | | KNCLTKLLPCFLEHNMKRDEDPHKAAKEMPFQGSGKSAWCPVEI |
| | | SKTVLWPESISVVRCVELFEAPVECEEEEEVEEEKGSFCASPESSRD |
| | | DFQEGREGIVARLTESLFLDLLGEENGGFCQQDMGESCLLPPSGST |
| | | SAHMPWDEFPSAGPKEAPPWGKEQPLHLEPSPPASPTQSPDNLTC |
| | | TETPLVIAGNPAYRSFSNSLSQSPCPRELGPDPLLARHLEEVEPEMP |
| | | CVPQLSEPTTVPQPEPETWEQILRRNVLQHGAAAAPVSAPTSGYQ |
| | | EFVHAVEQGGTQASAVVGLGPPGEAGYKAFSSLLASSAVSPEKCG |
| | | FGASSGEEGYKPFQDLIPGCPGDPAPVPVPLFTFGLDREPPRSPQSS |
| | | HLPSSSPEHLGLEPGEKVEDMPKPPLPQEQATDPLVDSLGSGIVYS |
| | | ALTCHLCGHLKQCHGQEDGGQTPVMASPCCGCCCGDRSSPPTTP |
| | | LRAPDPSPGGVPLEASLCPASLAPSGISEKSKSSSSFHPAPGNAQSS |
| | | SQTPKIVNFVSVGPTYMRVSERTMPRIPTLKNLEDLVTEYHGNFSA |
| | | WSGVSKGLAESLQPDYSERLCLVSEIPPKGGALGEGPGASPCNQH |
| | | SPYWAPPCYTLKPET |
| 27 | CD8(hinge/TM)- | TTTPAPRPPTPAPTIASQPLSLRPEACRPAAGGAVHTRGLDFACDIY |
| | 4-1BB- | IWAPLAGTCGVLLLSLVITLYCKRGRKKLLYIFKQPFMRPVQTTQE |
| | CD3zeta-IL9Ra- | EDGCSCRFPEEEEGGCELRVKFSRSADAPAYQQGQNQLYNELNLG |
| | IL2Rg | RREEYDVLDKRRGRDPEMGGKPRRKNPQEGLYNELQKDKMAEA |
| | | YSEIGMKGERRRGKGHDGLYQGLSTATKDTYDALHMQALPPRKL |
| | | SPRVKRIFYQNVPSPAMFFQPLYSVHNGNFQTWMGAHGAGVLLS |
| | | QDCAGTPQGALEPCVQEATALLTCGPARPWKSVALEEEQEGPGT |
| | | RLPGNLSSEDVLPAGCTEWRVQTLAYLPQEDWAPTSLTRPAPPDS |
| | | EGSRSSSSSSSSNNNNYCALGCYGGWHLSALPGNTQSSGPIPALAC |
| | | GLSCDHQGLETQQGVAWVLAGHCQRPGLHEDLQGMLLPSVLSK |
| | | ARSWTFERTMPRIPTLKNLEDLVTEYHGNFSAWSGVSKGLAESLQ |
| | | PDYSERLCLVSEIPPKGGALGEGPGASPCNQHSPYWAPPCYTLKPE |
| | | T |

In another aspect of the invention, it provides a method for preparing a CAR-T cell expressing the chimeric antigen receptor of the first aspect of the invention, wherein the method comprises the steps of: transducing the nucleic acid molecule or the vector of the instant invention into T cells, thereby obtaining the CAR-T cell.

In some embodiments, said method of preparing the aforesaid CAR-T cell comprising:

constructing a lentiviral vector comprising a nucleic acid encoding the chimeric antigen receptor as described in the present application;

isolating human peripheral blood mononuclear cells and purifying T cells therefrom;

inoculating the purified T cells to a culture plate under suitable stimulation conditions;

culturing the culture plate for a predetermined period of time;

after the predetermined period of time infecting the T cells with the lentiviral vector encoding the chimeric antigen receptor and subjecting to infected cells to cell expansion under suitable stimulator conditions to obtain the CAR-T cell expressing the chimeric antigen receptor as described in the present application.

The instant invention also provides a preparation comprising the aforesaid CAR-T cell. Further, said preparation further includes a pharmaceutically diluents or excipient.

In some embodiments, the preparation is a liquid preparation.

In some embodiments, the formulation of the preparation is an injection.

In some embodiments, the preparation comprises the host cell of the fourth aspect of the invention, and the concentration of the host cell is 1×10³-1×10⁸ cells/ml, preferably 1×10⁴-1×10⁷ cells/ml.

The instant invention also provides a method of preventing or treating a tumor, comprising administrating the aforesaid CAR-T cell to a subject in need thereof.

In some embodiments, the CAR-T cell is administered intravenously, subcutaneously, intramuscularly, intraperitoneally, or through spinal.

In some embodiments, the CAR-T cell is administered intravenously.

In some embodiments, the CAR-T cell is allogeneic or autologous.

In some embodiments, wherein the subject is a human.

In some embodiments, the tumor is selected from the group consisting of a hematological tumor, a solid tumor, and a combination thereof.

In some embodiments, the blood tumor is selected from the group consisting of acute myeloid leukemia (AML), multiple myeloma (MM), chronic lymphocytic leukemia (CLL), acute lymphoblastic leukemia (ALL), diffuse large B cell lymphoma (DLBCL), and a combination thereof.

In some embodiments, the solid tumor is selected from the group consisting of gastric cancer, peritoneal metastasis of gastric cancer, liver cancer, leukemia, renal cancer, lung cancer, small intestine cancer, bone cancer, prostate cancer, colorectal cancer, breast cancer, large intestine cancer, cervical cancer, ovarian cancer, lymphoma, nasopharyngeal carcinoma, adrenal tumor, bladder tumor, non-small cell lung cancer (NSCLC), glioma, endometrial cancer, and a combination thereof.

In another aspect of the invention, it provides a kit for the preparation of the cell of the fourth aspect of the invention, wherein the kit comprises a container, and the nucleic acid molecule of the second aspect of the invention or the vector of the third aspect of the invention located in the container.

As compared with the prior art, the present invention has the following beneficial effects:

said CAR-T cell of the present invention significantly increases the tumor killing ability and the expansion ability, and has significantly increased ability of killing solid/metastatic tumors. Said CAR-T cell of present invention includes a third signal receptor (IL2Ra, IL2Rb, IL4Ra, IL7Ra, IL9Ra, IL15Ra, IL21Ra, etc.), which is not a conventionally used ligand or excreted factor. For example, the third signal receptor IL7Ra is primarily expressed in memory CD4 and CD8 T cells, and plays an important role in the long-term survival of T cells and the formation of memory T cells. Integrating the third signal receptor signal into the CAR-T has a potential effect-enhancing function, and only works on the CAR-T cell, thereby reducing the risk of causing an immune side effect.

The present invention constructs a novel CAR-T cell including the third signal receptor, which increases the activation ability and survival ability of CAR-T cells in tumors as compared with the current CAR-T technology in clinic use. The activation ability and expansion ability of the cells are significantly enhanced, so that the CAR-T cell exhibits increased therapeutic effects and has more superior anti-tumor therapeutic effect.

### BRIEF DESCRIPTION OF THE DRAWINGS

FIG. 1 is an illustrative schematic view showing the molecular structure of chimeric antigen receptor (CAR) including a third signal receptor in embodiments of the present invention;

FIG. 2 is a schematic view showing the virus titer measured after 293 cells were infected with 20BBZIL2RbIL2Rg virus in an embodiment of the present invention.

FIG. 3 is a schematic view showing the virus titer measured after 293 cells were infected with 20BBZIL4RaIL2Rg virus in an embodiment of the present invention;

FIG. 4 is a schematic view showing the virus titer measured after 293 cells were infected with 20BBZIL7RaIL2Rg virus in an embodiment of the present invention;

FIG. 5 is a schematic view showing the virus titer measured after 293 cells were infected with 20BBZIL9RaIL2Rg virus in an embodiment of the present invention;

FIG. 6 is a schematic view showing the virus titer measured after 293 cells were infected with 20BBZIL21RaIL2Rg virus in an embodiment of the present invention;

FIG. 7 is a schematic view showing the results of phenotypic analysis of 20BBZ CAR-T cell and 20BBZIL2RbIL2Rg CAR-T cell in an embodiment of the present invention;

FIG. 8 is a schematic view showing the results of phenotypic analysis of 20BBZ CAR-T cell and 20BBZIL4RaIL2Rg CAR-T cell in an embodiment of the present invention;

FIG. 9 is a schematic view showing the results of phenotypic analysis of 20BBZ CAR-T cell and 20BBZIL7RaIL2Rg CAR-T cell in an embodiment of the present invention;

FIG. 10 is a schematic view showing the results of phenotypic analysis of 20BBZ CAR-T cell and 20BBZIL9RaIL2Rg CAR-T cell in an embodiment of the present invention;

FIG. 11 is a schematic view showing the results of phenotypic analysis of 20BBZ CAR-T cell and 20BBZIL21RaIL2Rg CAR-T cell in an embodiment of the present invention;

FIG. 12 is a schematic view showing the amplification ability of 20BBZ CAR-T cell and 20BBZIL7RaIL2Rg CAR-T cell in an embodiment of the present invention;

FIG. 13 is a schematic view showing the tumor killing ability of 20BBZ CAR-T cell, 20BBZIL21RaIL2Rg CAR-T cell, 20BBZIL9RaIL2Rg CAR-T cell and 20BBZIL7RaIL2Rg CAR-T cell in an embodiment of the present invention;

FIG. 14 is a schematic view showing the number of 20BBZ CAR-T cell and 20BBZIL7RaIL2Rg CAR-T cell in the bone marrow in an embodiment of the present invention;

FIG. 15 is a schematic view showing the in vivo survival ability of 20BBZ CAR-T cell and 20BBZIL7RaIL2Rg CAR-T cell in an embodiment of the present invention.

FIG. 16 is a schematic view showing the amplification ability of 19BBZ CAR-T cell and 19BBZIL7RaIL2Rg CAR-T cell in an embodiment of the present invention;

FIG. 17 is a schematic view showing the tumor killing ability of 19BBZ CAR-T and 19BBZIL7RaIL2Rg CAR-T cell in an embodiment of the present invention;

FIG. 18 is a schematic view showing the amplification ability of CLDN18.2BBZ CAR-T cell and CLDN18.2BBZIL21RaIL2Rg CAR-T cell in an embodiment of the present invention;

FIG. 19 is a schematic view showing the tumor killing ability of CLDN18.2BBZ CAR-T cell and CLDN18.2BBZIL21RaIL2Rg CAR-T cell in an embodiment of the present invention;

FIG. 20 is a schematic view showing the amplification ability of EGFRBBZ CAR-T cell and EGFRBBZIL21RaIL2Rg CAR-T cell in an embodiment of the present invention;

FIG. 21 is a schematic view showing the tumor killing ability of EGFRBBZ CAR-T cell and EGFRBBZIL21RaIL2Rg CAR-T cell in an embodiment of the present invention.

### TERMS

To make the disclosure easier to understand, some terms are firstly defined. As used in this application, unless expressly stated otherwise herein, each of the following terms shall have the meanings given below. Other definitions are set forth throughout the application.

As used herein, the term “about” may refer to a value or composition within an acceptable error range for a particular value or composition as determined by those skilled in the art, which will depend in part on how the value or composition is measured or determined.

As used herein, the term “administering” refers to the physical introduction of a product of the invention into a subject using any one of various methods and delivery systems known to those skilled in the art, including intravenous, intramuscular, subcutaneous, intraperitoneal, spinal or other parenteral administration, such as by injection or infusion.

As used herein, the term “antibody” (Ab) may comprise, but is not limited to, an immunoglobulin that specifically binds an antigen and contains at least two heavy (H) chains and two light (L) chains linked by disulfide bonds, or an antigen binding parts thereof. Each H chain contains a heavy chain variable region (abbreviated herein as VH) and a heavy chain constant region. The heavy chain constant region contains three constant domains, CH1 CH2, and CH3. Each light chain contains a light chain variable region (abbreviated herein as VL) and a light chain constant region. The light chain constant region contains a constant domain CL. The VH and VL regions can be further subdivided into hypervariable regions called complementarity determining regions (CDR), which are interspersed within more conservative regions called framework regions (FR). Each VH and VL contains three CDRs and four FRs, which are arranged from amino terminal to carboxy terminal in the following order: FR1, CDR1, FR2, CDR2, FR3, CDR3, and FR4. The variable regions of the heavy and light chains contain a binding domain that interacts with an antigen.

As used herein, the term “Single-chain Fv” also abbreviated as “scFv,” are antibody fragments that comprise the VH and VL antibody domains connected into a single polypeptide chain. In some embodiments, the scFv polypeptide further comprises a polypeptide linker between the VH and VL domains which enables the scFv to form the desired structure for antigen binding. For a review of scFv, see Plückthun in The Pharmacology of Monoclonal Antibodies, vol. 113, Rosenburg and Moore eds., Springer-Verlag, New York, pp. 269-315 (1994).

As used herein, the term “chimeric antigen receptor (CAR)” generally refers to an antigen receptor fused by fusing an antigen binding region of an antibody which recognizes a tumor associated antigen (TAA) or a binding fragment of other target molecules with an “immune receptor tyrosine-based activation motifs (ITAM, typically CD3ζ or FcεRIγ) of an intracellular signal domain. For example, the basic structure of CAR can include an antigen binding domain of a tumor-associated antigen (TAA) or other target molecules (typically, an scFv originated from the antigen binding region of a monoclonal antibody), an extracellular hinge region, a transmembrane region, and an immunoreceptor tyrosine-based activation motif (ITAM) of an intracellular immune receptor.

As used herein, the term “binding domain” generally refers to a domain that (specifically) binds to a given target epitope or a given target site of a target molecule (e.g., an antigen), interacts with the given target epitope or the given target site, or recognizes the given target epitope or the given target site.

As used herein, the term “specific binding” generally refers to a measurable and reproducible interaction, such as, the binding between a target and an antibody, which can determine the presence of a target in the presence of heterogeneous populations of molecules (including biomolecules). For example, antibodies that specifically bind to targets (which can be epitopes) are antibodies that bind the target(s) with greater compatibility, affinity, easiness, and/or duration than other targets. In some embodiments, the antibody specifically binds to an epitope on a protein that is conserved in proteins of different species. In another embodiment, the specific binding includes but is not limited to exclusive binding.

As used herein, the term “transmembrane domain” generally refers to a polypeptide or protein which is encoded at a DNA level by an exon including at least an extracellular region, a transmembrane region, and an intracellular region. The transmembrane domain generally includes three different structural regions: N-terminal extracellular region, middle conserved transmembrane extension region, and C-terminal cytoplasmic region. The transmembrane domain may further include an intracellular region or a cytoplasmic region.

As used herein, the term “hinge region” generally refers to a region located between the binding domain and the transmembrane domain in the CAR structure. The hinge region usually comes from IgG family, such as IgG1 and IgG4, and some from IgD and CD8. Generally, the hinge region has a certain degree of flexibility, which affects the spatial constraints between the CAR molecule and its specific target, thereby affecting the contact between CAR T cells and tumor cells.

As used herein, the term “costimulatory” generally refers to a source of the second signal of lymphocyte activation, which is usually generated by an interaction of costimulatory molecules on the surface of immune cells (between T cells/B cells or between antigen presenting cells/T cells) involved in adaptive immunity with their receptors. For example, the complete activation of T cells depends on dual signaling and the action of cytokine. The first signal of T cell activation is derived from the specific binding of its receptors with the antigens, that is, the recognition of T cells to the antigens; and the second signal of T cell activation is derived from the costimulatory molecule, that is, the interaction of the costimulatory molecules of the antigen presenting cells with the corresponding receptors on the surfaces of T cells.

As used herein, the term “costimulatory domain” generally refers to an intracellular portion of the corresponding receptor of the costimulatory molecule, which can transduce a costimulatory signal (also known as the second signal). For example, in CAR-T cells, the costimulatory domain derived from CD137 (or receptors of other costimulatory molecules) can be activated after the binding of the extracellular binding domain in the CAR structure with the corresponding antigen, thereby transducing a costimulatory signal.

As used herein, the term “primary signal transduction domain” generally refers to an amino acid sequence within a cell that can generate signals which promote the immune effector function of CAR-containing cells such as CAR-T cells. Examples of the immune effector functions in, e.g., CAR-T cells can include cell lysis activity and auxiliary activity, including cytokine secretion. In some embodiments, the primary signal transduction domain transduces the effector functional signals and directs the cells to perform the specialization function. Although the primary signal transduction domain can be used in its entirety, it is not necessary to use the entire chain in many cases. As for the use of a truncated portion of the primary signal transduction domain, such truncated portion can be used to replace the intact chain, as long as it can transduce the effector functional signals. The term “primary signal transduction domain” is thus intended to encompass any truncated portion of an intracellular signal transduction domain that is sufficient to transduce the effector functional signals. For example, in CAR-T cells, the primary signal transduction domain derived from CD3 zeta.

As used herein, the term “tumor” generally refers to a neoplasm or solid lesion formed by abnormal cell growth. In the present application, the tumor can be a solid tumor or a non-solid tumor. In some embodiments, a visible lump that can be detected by clinical examinations such as, X-ray radiography, CT scanning, B-ultrasound or palpation can be called solid tumor, while a tumor that cannot be seen or touched by X-ray, CT scanning, B-ultrasound and palpation, such as leukemia, can be called non-solid tumor.

As used herein, the term “pharmaceutically acceptable diluent” or “pharmaceutically acceptable excipient” generally refers to a pharmaceutically acceptable substance, composition, or vehicle involved in carrying, storing, transferring, or administering a cell preparation, e.g., liquids, semi-solid or solid fillers, diluents, osmotic agents, solvent, or encapsulating substances. The pharmaceutically acceptable diluent or excipient can include a pharmaceutically acceptable salt, wherein the term “pharmaceutically acceptable salt” includes salts of active compounds prepared by using a relatively nontoxic acid or base, e.g., sodium chloride, depending on the cell nature of the present application. The pharmaceutically acceptable carrier can further include organic acids (e.g., lactic acid), bioactive substances (e.g., polypeptides, antibodies, and the like) and antibiotics (e.g., penicillin, streptomycin), etc. The pharmaceutically acceptable carrier can further include a hydrogel, such as, a hydrogel containing polyacrylamide. The pharmaceutically acceptable diluent or excipient can include storage solution, cryopreservation solution, injection, etc., which can be used for cells. In general, the pharmaceutically acceptable diluent or excipient can maintain the activity of the cells carried by the carrier without hindering its therapeutic efficacy. The pharmaceutically acceptable diluent or excipient can also contribute to the storage, transportation, proliferation and migration of cells, and is suitable for clinical application.

As used herein, the term “subject” generally refers to a human or non-human animal, including but not limited to a cat, dog, horse, pig, cow, sheep, rabbit, mouse, rat, or monkey. In some embodiments, said subject is a human.

As used herein, the term “include/including” or “comprise/comprising” generally refers to encompassing clearly specified features, but does not exclude other elements.

### DETAILED DESCRIPTION OF THE EMBODIMENTS

The present invention provides a chimeric antigen receptor including a third signal receptor, and said chimeric antigen receptor have a structure of scFv(X)-(Y)CD3zeta-MN; wherein X is a tumor-targeting antibody or other protein; Y is the intracellular domain of costimulatory receptor, said costimulatory receptor is selected from ICOS, CD28, CD27, HVEM, LIGHT, CD40L, 4-1BB, OX40, DR3, GITR, CD30, TIM1, SLAM, CD2, CD226; M is the intracellular domain of gamma chain family cytokine receptor, said cytokine receptor is selected from IL2Ra, IL2Rb, IL4Ra, IL7Ra, IL9Ra, IL15Ra, IL21Ra; and N is the intracellular domain of IL2Rg. The present invention also relates to a CAR-T cell constructed with a recombinant expression vector of any one of the aforesaid chimeric antigen receptor and a preparation method therefor, a formulation including the CAR-T cell, and use of the CAR-T cell.

Hereinafter the embodiments of the present invention are further described with reference to the accompanying drawings and examples. The following examples are only for more clearly illustrating the technical solutions of the present invention, but not for limiting the protective scope of the present invention.

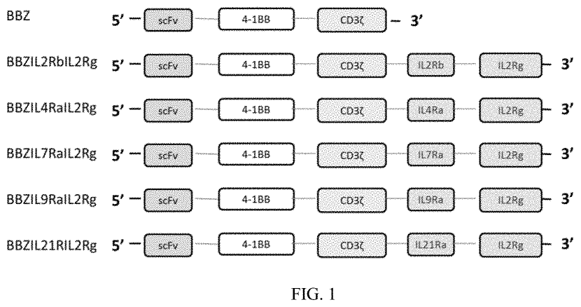

The chimeric antigen receptors (CAR) including the third signal receptor used in the examples of the present invention are BBZIL2RbIL2Rg, BBZIL4RaIL2Rg, BBZIL7RaIL2Rg, BBZIL9RaIL2Rg, BBZIL21RaIL2Rg, respectively, and their structures are shown in FIG. 1.

### EXAMPLE 1—Preparation of 20BBZIL2RbIL2Rg CAR-T Cell

The preparation of said 20BBZIL2RbIL2Rg CAR-T cell in this example includes the following steps:

1. Construction of Lentiviral Vector pCDH-MSCVEF-20BBZIL2RbIL2Rg and Production of Virus

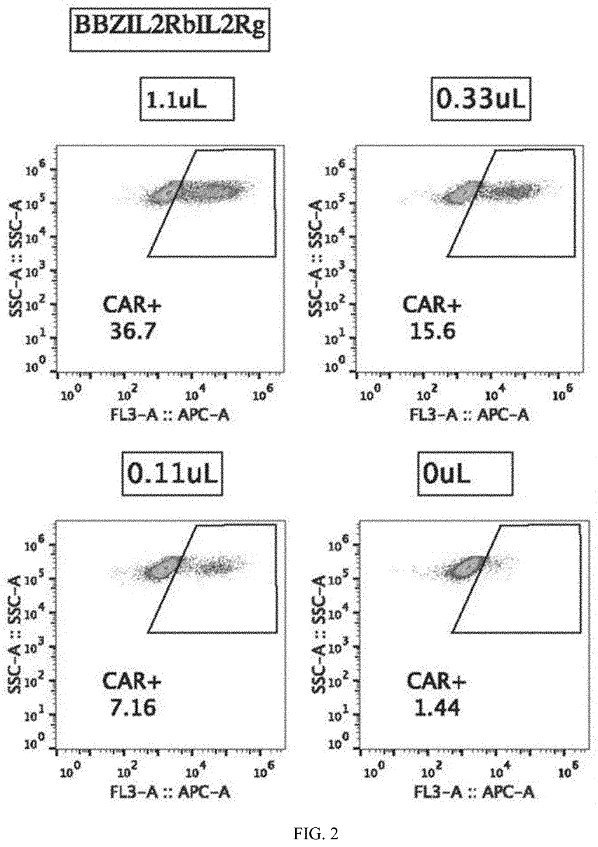

Forming a fusion protein of scFv-antihCD20-20BBZ (SEQ ID No:1), IL2Rb intracellular domain (SEQ ID No:3) and the intracellular domain of IL2Rg (SEQ ID No:7) by overlap PCR, and adding EcoRI and BamHI restriction sites to both ends of the fusion protein to clone a pCDH-MSCVEF vector. Subjecting the clones sequenced correctly to a large scale endotoxin-free extraction, and co-transfecting with lentiviral packaging plasmid (VSV-g, pMD Gag/Pol, RSV-REV) into 293×. After 48 and 72 hours, collecting the supernatant, filtering it with a 0.45 μm filter, and centrifuging with Beckman ultracentrifuge and SW28 head at 25000 RPM for 2 hours to concentrate the virus, that is, the pCDH-MSCVEF-20BBZIL2RbIL2Rg virus (briefly, 20BBZIL2RbIL2Rg virus) for use in the subsequent production of CAR-T cells. Meanwhile, producing the control pCDH-MSCVEF-20BBZ virus (briefly, 20BBZ virus), and infecting 293 cells with the obtained 20BBZIL2RbIL2Rg virus to determine the virus titer, as shown in FIG. 2.

2. Preparation of 20BBZIL2RbIL2Rg CAR-T Cell and 20BBZ CAR-T Cell

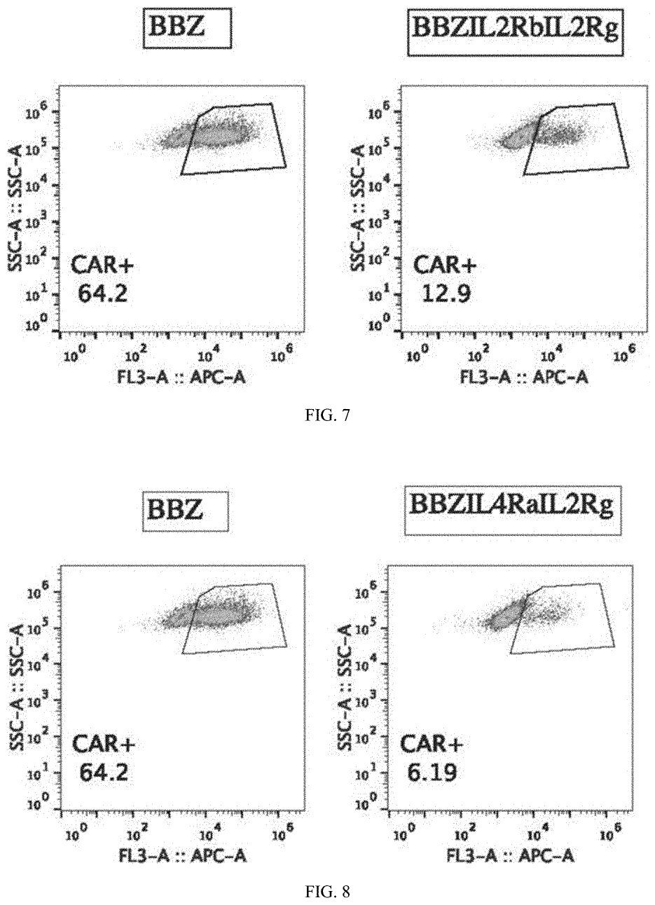

Purifying human PBMC with a Stemcell T cell isolation kit, inoculating into a 96-well culture plate coated by anti-hCD3 and anti-hCD28. After 2 days, infecting the cells with 20BBZ virus and 20BBZIL2RbIL2Rg virus at MOI=10-20. After 1 day, continuing to culture the cells with the medium changed, and stimulating with artificial antigen presenting cell or anti-hCD3/28 every 6 days. After 2 rounds of stimulation, the obtained cells are 20BBZ CAR-T cell and 20BBZIL2RbIL2Rg CAR-T cell for use in the subsequent experiments and phenotypic analysis. As shown in FIG. 7, the obtained cells are CAR-positive.

### EXAMPLE 2—Preparation of 20BBZIL4RaIL2Rg CAR-T cell

The preparation of said 20BBZIL4RaIL2Rg CAR-T cell in this example includes the following steps:

1. Construction of Lentiviral Vector pCDH-MSCVEF-20BBZIL4RaIL2Rg and Production of Virus

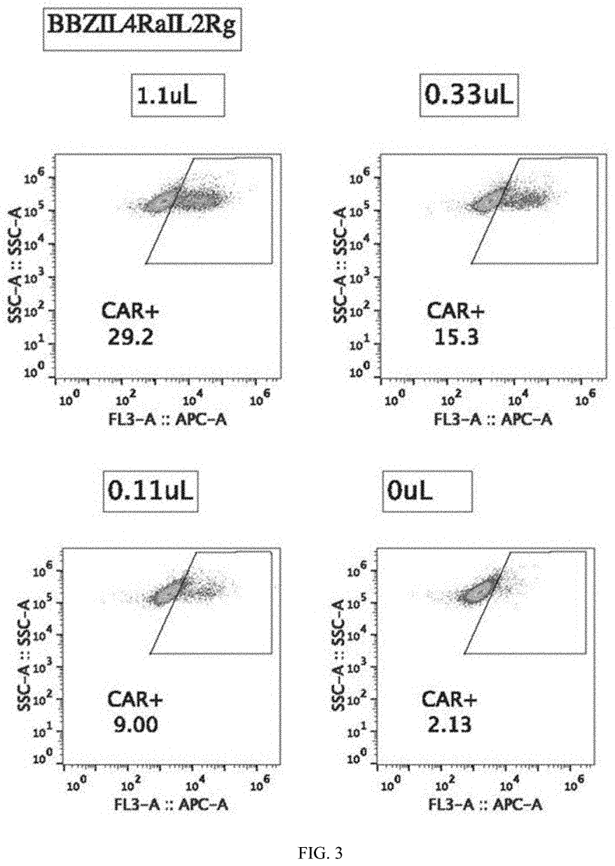

Forming a fusion protein of scFv-antihCD20-20BBZ (SEQ ID No:1), IL4Ra intracellular domain (SEQ ID No:4) and the intracellular domain of IL2Rg (SEQ ID No:7) by overlap PCR, and adding EcoRI and BamHI restriction sites to both ends of the fusion protein to clone a pCDH-MSCVEF vector. Subjecting the clones sequenced correctly to a large scale endotoxin-free extraction, and co-transfecting with lentiviral packaging plasmid (VSV-g, pMD Gag/Pol, RSV-REV) into 293×. After 48 and 72 hours, collecting the supernatant, filtering it with a 0.45 μm filter, and centrifuging with Beckman ultracentrifuge and SW28 head at 25000 RPM for 2 hours to concentrate the viruses, that is, the pCDH-MSCVEF-20BBZIL4RaIL2Rg virus (briefly, 20BBZIL4RaIL2Rg virus) for use in the subsequent production of CAR-T cells. Meanwhile, producing the control pCDH-MSCVEF-20BBZ virus (briefly, 20BBZ virus), and infecting 293 cells with the obtained 20BBZIL4RaIL2Rg virus to determine the virus titer, as shown in FIG. 3.

2. Preparation of 20BBZIL4RaIL2Rg CAR-T Cell and 20BBZ CAR-T Cell

Purifying human PBMC with a Stemcell T cell isolation kit, inoculating into a 96-well culture plate coated by anti-hCD3 and anti-hCD28. After 2 days, infecting the cells with 20BBZ virus and 20BBZIL4RaIL2Rg virus at MOI=10-20. After 1 day, continuing to culture the cells with the medium changed, and stimulated by artificial antigen presenting cell or anti-hCD3/28 every 6 days. After 2 rounds of stimulation, the obtained cells are 20BBZCAR-T cell and 20BBZIL4RaIL2RgCAR-T cell for use in the subsequent experiments and phenotypic analysis. As shown in FIG. 8, the obtained cells are CAR-positive.

### EXAMPLE 3—Preparation of 20BBZIL7RaIL2Rg CAR-T cell

The preparation of said 20BBZIL7RaIL2Rg CAR-T cell in this example includes the following steps:

1. Construction of Lentiviral Vector pCDH-MSCVEF-20BBZIL7RaIL2Rg and Production of Virus

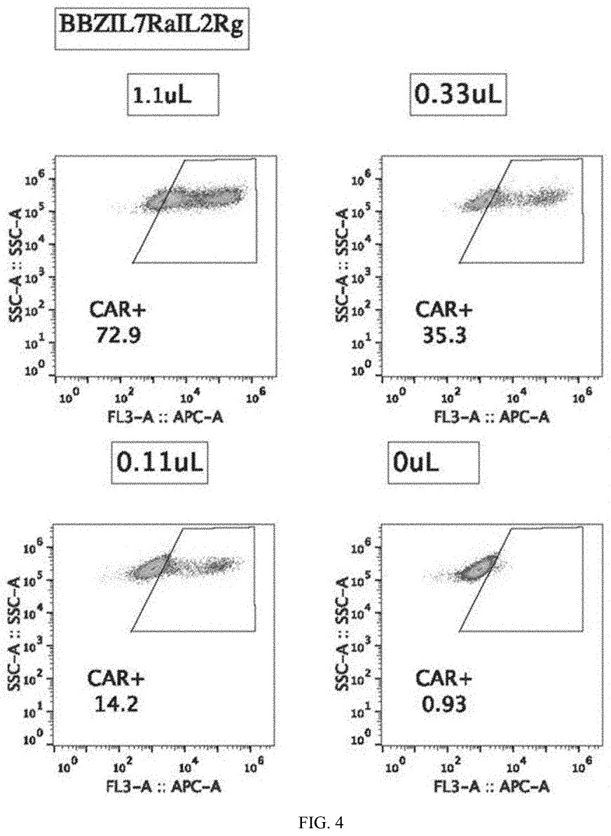

Forming a fusion protein of scFv-antihCD20-20BBZ (SEQ ID No:1), IL7Ra intracellular domain (SEQ ID No:2) and the intracellular domain of IL2Rg (SEQ ID No:7) by overlap PCR, and adding EcoRI and BamHI restriction sites to both ends of the fusion protein to clone a pCDH-MSCVEF vector. Subjecting the clones sequenced correctly to a large scale endotoxin-free extraction, and co-transfecting with lentiviral packaging plasmid (VSV-g, pMD Gag/Pol, RSV-REV) into 293×. After 48 and 72 hours, collecting the supernatant, filtering it with a 0.45 μm filter, and centrifuged with Beckman ultracentrifuge and SW28 head at 25000 RPM for 2 hours to concentrate the viruses, that is, the pCDH-MSCVEF-20BBZIL7RaIL2Rg virus (briefly, 20BBZIL7RaIL2Rg virus) for use in the subsequent production of CAR-T cells. Meanwhile, producing the control pCDH-MSCVEF-20BBZ virus (briefly, 20BBZ virus), and infecting 293 cells with 20BBZIL7RaIL2Rg virus to determine the virus titer, as shown in FIG. 4.

2. Preparation of 20BBZIL7RaIL2Rg CAR-T Cell and 20BBZ CAR-T Cell

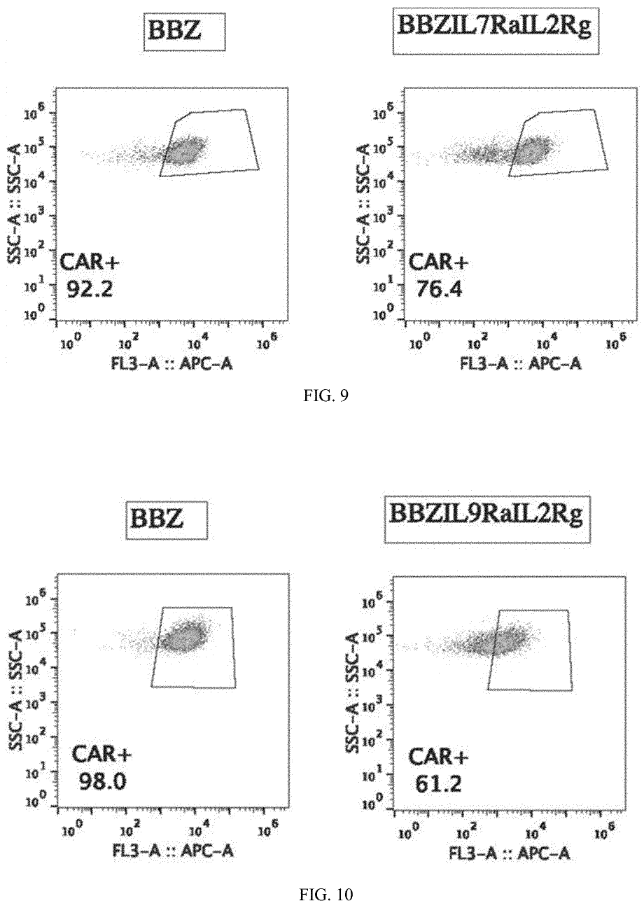

Purifying human PBMC with a Stemcell T cell isolation kit, inoculating into a 96-well culture plate coated by anti-hCD3 and anti-hCD28. After 2 days, infecting the cells with 20BBZ virus and 20BBZIL7RaIL2Rg virus at MOI=10-20. After 1 day, continuing to culture the cells with the medium changed, and stimulating with artificial antigen presenting cell or anti-hCD3/28 every 6 days. After 2 rounds of stimulation, the obtained cells are 20BBZCAR-T cell and 20BBZIL7RaIL2RgCAR-T cell for use in the subsequent experiments and phenotypic analysis. As shown in FIG. 9, the obtained cells are CAR-positive.

### EXAMPLE 4—Preparation of 20BBZIL9RaIL2Rg CAR-T cell

The preparation of said 20BBZIL9RaIL2Rg CAR-T cell in this example includes the following steps:

1. Construction of Lentiviral Vector pCDH-MSCVEF-20BBZIL9RaIL2Rg and Production of Virus

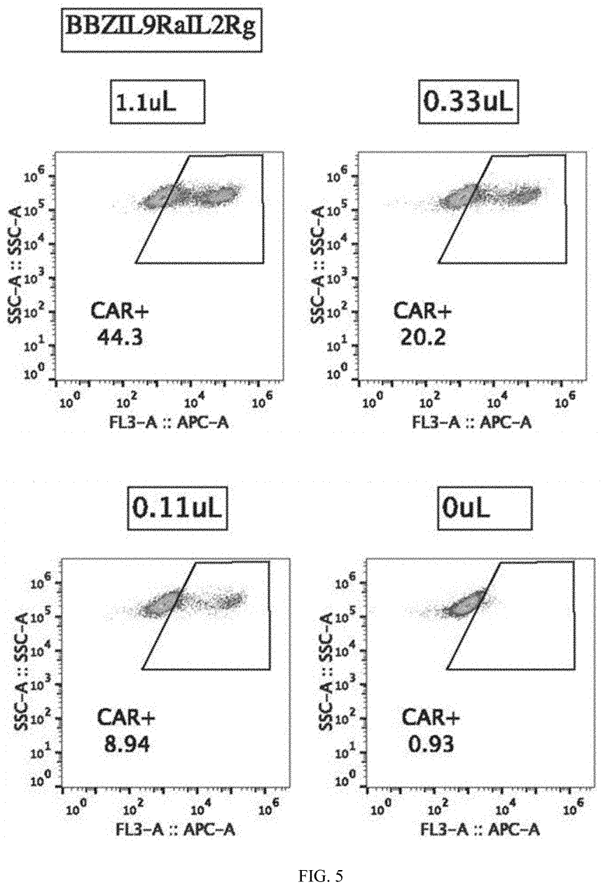

Forming a fusion protein of scFv-antihCD20-20BBZ (SEQ ID No:1), IL9Ra intracellular domain (SEQ ID No:5) and the intracellular domain of IL2Rg (SEQ ID No:7) by overlap PCR, and adding EcoRI and BamHI restriction sites to both ends of the fusion protein to clone a pCDH-MSCVEF vector. Subjecting the clones sequenced correctly to a large scale endotoxin-free extraction, and co-transfecting with lentiviral packaging plasmid (VSV-g, pMD Gag/Pol, RSV-REV) into 293×. After 48 and 72 hours, collecting the supernatant, filtering it with a 0.45 μm filter, and centrifuging with Beckman ultracentrifuge and SW28 head at 25000 RPM for 2 hours to concentrate the viruses, that is, the pCDH-MSCVEF-20BBZIL9RaIL2Rg virus (briefly, 20BBZIL9RaIL2Rg virus) for use in the subsequent production of CAR-T cells. Meanwhile, producing the control pCDH-MSCVEF-20BBZ virus (briefly, 20BBZ virus), and infecting 293 cells with 20BBZIL9RaIL2Rg virus to determine the virus titer, as shown in FIG. 5.

2. Preparation of 20BBZIL9RaIL2Rg CAR-T Cell and 20BBZ CAR-T Cell

Purifying human PBMC with a Stemcell T cell isolation kit, inoculating into a 96-well culture plate coated by anti-hCD3 and anti-hCD28. After 2 days, infecting the cells with 20BBZ virus and 20BBZIL9RaIL2Rg virus at MOI=10-20. After 1 day, continuing to culture the cells with the medium changed, and stimulating with artificial antigen presenting cell or anti-hCD3/28 every 6 days. After 2 rounds of stimulation, the obtained cells are 20BBZCAR-T cell and 20BBZIL9RaIL2RgCAR-T cell for use in the subsequent experiments and phenotypic analysis. As shown in FIG. 10, the obtained cells are CAR-positive.

### EXAMPLE 5—Preparation of 20BBZIL21RaIL2Rg CAR-T cell

The preparation of the 20BBZIL21RaIL2Rg CAR-T cell in this example includes the following steps:

1. Construction of Lentiviral Vector pCDH-MSCVEF-20BBZIL21RaIL2Rg and Production of Virus

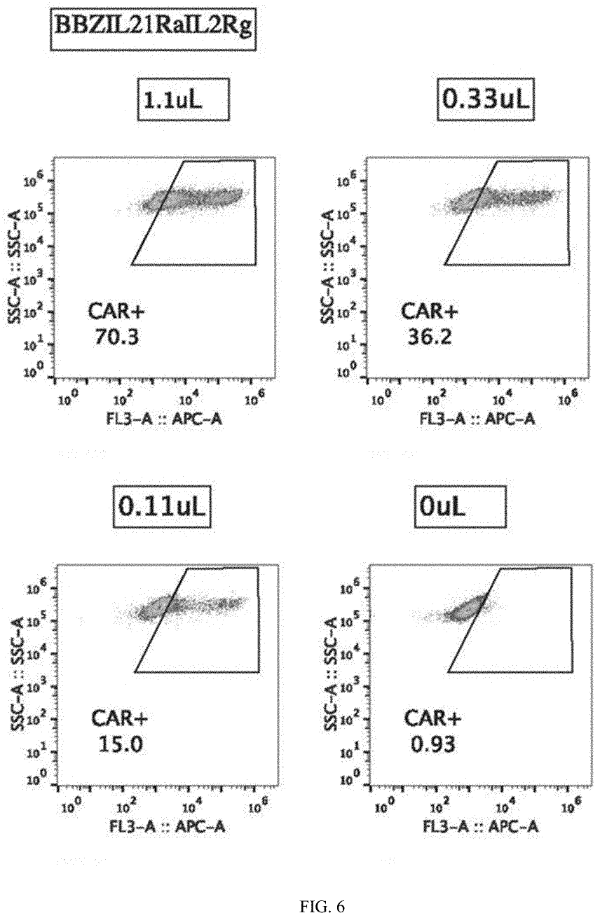

Forming a fusion protein of scFv-antihCD20-20BBZ (SEQ ID No:1), IL21Ra intracellular domain (SEQ ID No:6) and the intracellular domain of IL2Rg (SEQ ID No:7) by overlap PCR, and adding EcoRI and BamHI restriction sites to both ends of the fusion protein to clone a pCDH-MSCVEF vector. Subjecting the clones sequenced correctly to a large scale endotoxin-free extraction, and co-transfecting with lentiviral packaging plasmid (VSV-g, pMD Gag/Pol, RSV-REV) into 293×. After 48 and 72 hours, collecting the supernatant, filtering it with a 0.45 uM filter, and centrifuging with Beckman ultracentrifuge and SW28 head at 25000 RPM for 2 hours to concentrate the viruses, that is, the pCDH-MSCVEF-20BBZIL21RaIL2Rg virus (briefly, 20BBZIL21RaIL2Rg virus) for use in the subsequent production of CAR-T cells. Meanwhile, producing the control pCDH-MSCVEF-20BBZ virus (briefly, 20BBZ virus), and infecting 293 cells with 20BBZIL21RaIL2Rg virus to determine the virus titer, as shown in FIG. 6.

2. Preparation of 20BBZIL21RaIL2Rg CAR-T Cell and 20BBZ CAR-T Cell

Purifying human PBMC with a Stemcell T cell isolation kit, inoculating into a 96-well culture plate coated by anti-hCD3 and anti-hCD28. After 2 days, infecting the cells with 20BBZ virus and 20BBZIL21RaIL2Rg virus at MOI=10-20. After 1 day, continuing to culture the cells with the medium changed, and stimulating by artificial antigen presenting cell or anti-hCD3/28 every 6 days. After 2 rounds of stimulation, the obtained cells are 20BBZCAR-T cell and 20BBZIL21RaIL2RgCAR-T cell for use in the subsequent experiments and phenotypic analysis. As shown in FIG. 11, the obtained cells are CAR-positive.

### EXAMPLE 6—Comparison of Expansion Abilities of 20BBZ CAR-T Cell and 20BBZIL7RaIL2Rg CAR-T Cell

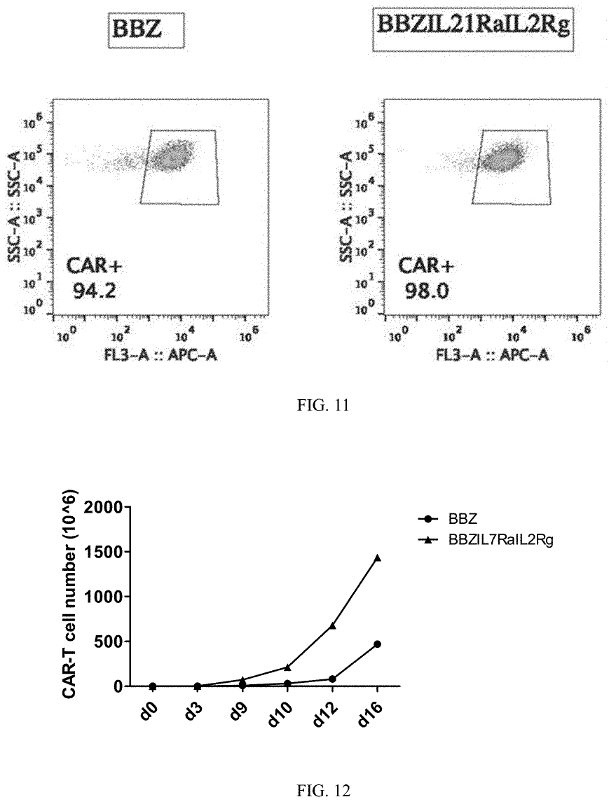

Culture the 20BBZ CAR-T cell and 20BBZIL7RaIL2Rg CAR-T cell prepared in Step 2 of Example 3 continuously for 14 days, and stimulate with artificial antigen presenting cell once every 6 days. Count the cells, and the results are shown in FIG. 12. It can be seen from the figure that 20BBZIL7RaIL2Rg CAR-T cell has enhanced proliferation ability as compared with 20BBZCAR-T cell.

### EXAMPLE 7—Comparison of Tumor-Killing Abilities of 20BBZ CAR-T Cell and 20BBZIL7RaIL2Rg CAR-T Cell

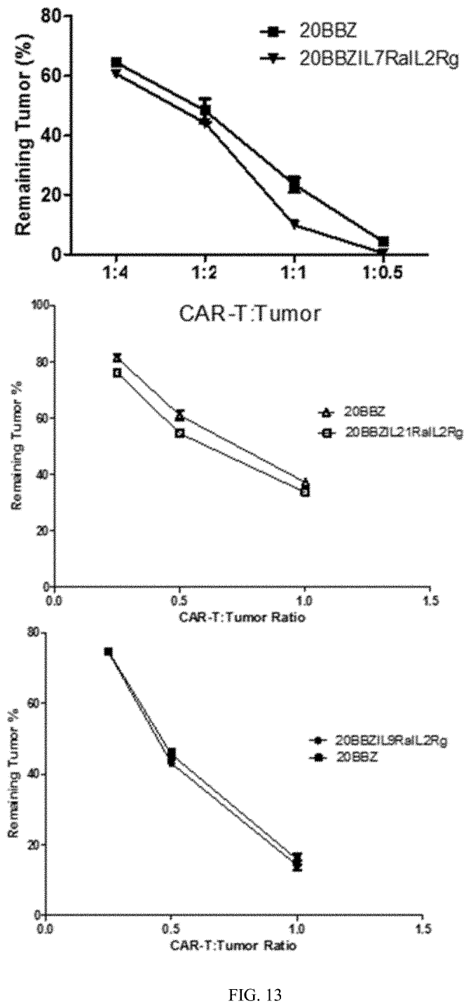

Inoculate the 20BBZ CAR-T cell, 20BBZIL21RaIL2Rg CAR-T cell, 20BBZIL9RaIL2Rg CAR-T cell and 20BBZIL7RaIL2Rg CAR-T cell prepared in Step 2 of Example 3 into a 96-well plate, and add the Raji tumor cells at a CAR-T:tumor cell ratio of 1:1, 1:2, 1:4. After 24 and 48 hours, compare the survival rates of tumor cells, and the results are shown in FIG. 13. It can be seen from the figure that the 20BBZIL21RaIL2Rg CAR-T cell, 20BBZIL9RaIL2Rg CAR-T cell and 20BBZIL7RaIL2Rg CAR-T cell has similar tumor killing ability as compared with 20BBZ CAR-T cell.

### EXAMPLE 8—Comparison of Anti-Tumor Ability and In Vivo Survival Ability of 20BBZ CAR-T Cell and 20BBZIL7RaIL2Rg CAR-T Cell

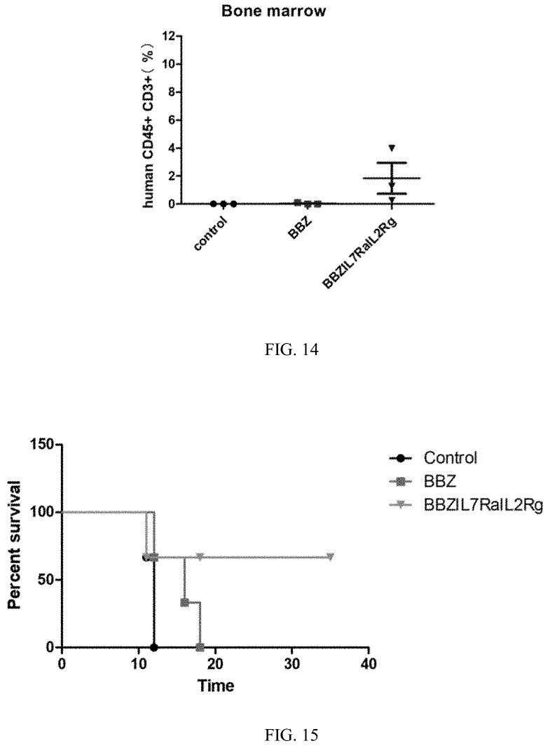

Inoculated 10⁶ Nalm-6 tumor cells intravenously into B-NDG mice. Treated the mice with 10⁷ 2 OBBZ CAR-T cells and 20BBZIL7RaIL2Rg CAR-T cells after 6 days. The mice were observed for their survival rates, and some mice were detected for the level of tumor cells and CAR-T cells in their marrow on Day 7. The results are shown in FIG. 14 and FIG. 15, respectively. It can be seen from the figures that 20BBZIL7RaIL2Rg CAR-T cell, as compared with 20BBZ CAR-T cell, substantially prolongs the survival of mice, and expanded more in vivo.

### Example 9—Preparation of 19BBZIL7RaIL2Rg CAR-T Cells

The preparation of the 19BBZIL7RaIL2Rg CAR-T cells described in this Example comprises the following steps:

1. Construction of the Lentiviral Vector pCDH-MSCVEF-19BBZIL7RaIL2Rg and Virus Production

Similar to Example 3, construct the scFv-antihCD19-BBZ (SEQ ID No: 17), IL7Ra intracellular region (SEQ ID No. 8) and IL2Rg intracellular region (SEQ ID No. 13) CAR plasmids and produce the lentivirus (referred to as 19BBZIL7RaIL2Rg virus) for subsequent CAR-T cell production. A control pCDH-MSCVEF-19BBZ virus (referred to as 19BBZ virus) was also produced.

2. Preparation of 19BBZIL7RaIL2Rg CAR-T Cells and 19BBZ CAR-T Cells

Human PBMC were inoculated into anti-hCD3 and anti-hCD28-coated 96-well culture plates, and after 2 days, the cells were infected with 19BBZ virus and 19BBZIL7RaIL2Rg virus according to MOI=10-20, and the cell culture was continued after 1 day of fluid change, and the resulting cells were 19BBZ CAR-T cells and 19BBZIL7RaIL2RgCAR-T cells, according to every 6 days using artificial antigen-presenting cells or anti-hCD3/28 stimulation, after a total of 2 rounds of stimulation to expand cells, the cells of the expansion process species were used for proliferation and killing comparative experiments.

### Example 10—Comparison of the Expansion Capacity of 19BBZ CAR-T Cells and 19BBZIL7RaIL2Rg CAR-T Cells

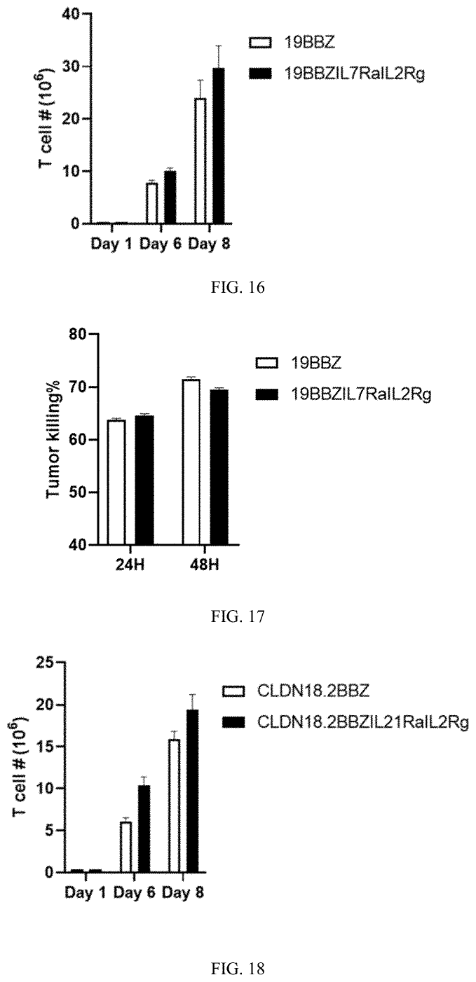

The 19BBZ CAR-T cells and 19BBZIL7RaIL2Rg CAR-T cells prepared in step 2 of Example 9 were cultured continuously for 12 days, and the cells were stimulated with artificial antigen presenting cells every 6 days and the cells were counted, and the results are shown in FIG. 16. From the FIG. 16, it can be seen that 19BBZIL7RaIL2Rg CAR-T cells have stronger proliferation ability relative to 19BBZCAR-T cells.

### Example 11—Comparison of Tumor Killing Ability of 19BBZ CAR-T Cells and 19BBZIL7RaIL2Rg CAR-T Cells

The 19BBZ CAR-T cells and 19BBZIL7RaIL2Rg CAR-T cells prepared in step 2 of Example 9 were inoculated into 96-well plates, and Raji tumor cells were added according to the CAR-T:Raji tumor cell ratio of 1:0.5, and the survival ratio of tumor cells was compared after 24 and 48 hours, and the results are shown in FIG. 17. From the figure, it can be seen that 19BBZIL7RaIL2Rg CAR-T cells have similar tumor killing ability relative to 19BBZ CAR-T cells.

### Example 12—Preparation of CLDN18.2BBZIL21RaIL2Rg CAR-T Cells

The preparation of the CLDN18.2BBZIL21RaIL2Rg CAR-T cells described in this Example comprises the following steps.

1. Construction of Lentiviral Vector pCDH-MSCVEF-CLDN18.2BBZIL21RaIL2Rg and Virus Production

Similar to Example 3, the scFv-antihCLDN18.2-BBZ (SEQ ID No: 19), IL21Ra intracellular region (SEQ ID No. 12) and IL2Rg intracellular region (SEQ ID No. 13) CAR plasmids were constructed and lentiviruses (referred to as CLDN18.2BBZIL21RaIL2Rg viruses) were produced for subsequent CAR-T cell production. A control pCDH-MSCVEF-CLDN18.2BBZ virus (referred to as CLDN18.2BBZ virus) was also produced.

2. Preparation of CLDN18.2BBZIL21RaIL2Rg CAR-T Cells and CLDN18.2BBZ CAR-T Cells

Human PBMC were inoculated into anti-hCD3 and anti-hCD28-coated 96-well culture plates, and after 2 days, infected with CLDN18.2BBZ virus and CLDN18.2BBZIL7RaIL2Rg virus according to MOI=10-20, and continued cell culture after 1 day of fluid change, and the resulting cells were CLDN18.2BBZ CAR-T cells and CLDN18.2BBZIL21RaIL2RgCAR-T cells, and the cells were expanded after a total of 2 rounds of stimulation using artificial antigen presenting cells or anti-hCD3/28 stimulation every 6 days, and the cells of the expansion process species were used for proliferation and killing comparison experiments.

### Example 13—Comparison of the Expansion Capacity of CLDN18.2BBZ CAR-T Cells and CLDN18.2BBZIL21RaIL2Rg CAR-T Cells

The CLDN18.2BBZ CAR-T cells and CLDN18.2BBZIL21RaIL2Rg CAR-T cells prepared in step 2 of Example 12 were cultured continuously for 12 days, and the cells were stimulated with artificial antigen-presenting cells every 6 days and the results were counted as shown in FIG. 18. From the FIG. 18, it is clear that CLDN18.2BBZIL21RaIL2Rg CAR-T cells have stronger proliferation ability relative to CLDN18.2BBZ CAR-T cells.

### Example 14—Comparing the Tumor Killing Ability of CLDN18.2BBZ CAR-T Cells and CLDN18.2BBZIL21RaIL2Rg CAR-T Cells

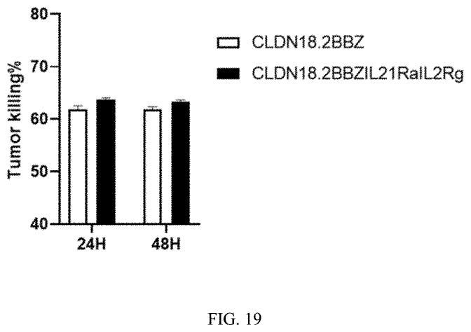

The CLDN18.2BBZ CAR-T cells and CLDN18.2BBZIL21RaIL2Rg CAR-T cells prepared in step 2 of Example 12 were inoculated into 96-well plates, and Raji tumor cells were added according to the CAR-T:Raji tumor cell ratio of 1:0.5, and the survival ratio of tumor cells was compared after 24 and 48 hours, and the results are shown in FIG. 19. From the FIG. 19, it can be seen that CLDN18.2BBZIL21RaIL2Rg CAR-T cells have a slightly elevated tumor killing ability relative to CLDN18.2BBZ CAR-T cells.

### Example 15—Preparation of EGFRBBZIL21RaIL2Rg CAR-T Cells

The preparation of EGFRBBZIL21RaIL2Rg CAR-T cells described in this Example comprises the following steps.

1. Construction of the Lentiviral Vector pCDH-MSCVEF-EGFRBBZIL21RaIL2Rg and Viral Production

Similar to Example 3, the scFv-anti-hEGFR-BBZ (SEQ ID No. 21), IL21Ra intracellular region (SEQ ID No. 12) and IL2Rg intracellular region (SEQ ID No. 13) CAR plasmids were constructed and lentiviruses (referred to as EGFRBBZIL21RaIL2Rg viruses) were produced for subsequent CAR-T cell production. A control pCDH-MSCVEF-EGFRBBZ virus (referred to as EGFRBBZ virus) was also produced.

2. Preparation of EGFRBBZIL21RaIL2Rg CAR-T Cells and EGFRBBZ CAR-T Cells

Human PBMC were inoculated into anti-hCD3 and anti-hCD28-coated 96-well culture plates, and after 2 days, infected with EGFRBBZ virus and EGFRBBZIL21RaIL2Rg virus according to MOI=10-20, and continued cell culture by changing the liquid after 1 day, and the resulting cells were EGFRBBZ CAR-T cells and EGFRBBZIL21RaIL2RgCAR-T cells, according to every 6 days using artificial antigen-presenting cells or anti-hCD3/28 stimulation, after a total of 2 rounds of stimulation to expand cells, the cells of the expansion process species were used for proliferation and killing comparative experiments.

### Example 16—Comparison of the Expansion Capacity of EGFRBBZ CAR-T Cells and EGFRBBZIL21RaIL2Rg CAR-T Cells

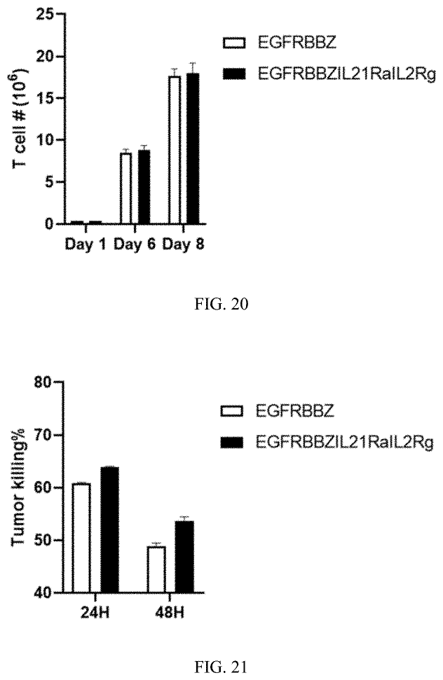

The EGFRBBZ CAR-T cells and EGFRBBZIL21RaIL2Rg CAR-T cells prepared in step 2 of Example 15 were cultured continuously for 12 days, and the cells were stimulated with artificial antigen presenting cells every 6 days and the results were counted as shown in FIG. 20. From the FIG. 20, it can be seen that EGFRBBZIL21RaIL2Rg CAR-T cells have stronger proliferation ability relative to EGFRBBZCAR-T cells.

### Example 17—Comparison of the Tumor Killing Ability of EGFRBBZ CAR-T Cells and EGFRBBZIL21RaIL2Rg CAR-T Cells

EGFRBBZ CAR-T cells and EGFRBBZIL21RaIL2Rg CAR-T cells prepared in step 2 of Example 15 were inoculated into 96-well plates, and Raji tumor cells were added according to the CAR-T:Raji tumor cell ratio of 1:0.5, and the survival ratio of tumor cells was compared after 24 and 48 hours, and the results are shown in FIG. 21. From the FIG. 21, it can be seen that EGFRBBZIL21RaIL2Rg CAR-T cells have a slightly elevated tumor killing ability relative to EGFRBBZ CAR-T cells.

It can be seen from the aforesaid examples that the present invention constructs a novel CAR-T cells including a third signal receptor, which significantly increases the activation ability, survival ability, expansion ability of the CAR-T cells in tumors, as compared with the current CAR-T technology in clinic use, and has more superior anti-tumor therapeutic effect.

Hereinbefore the specific embodiments of the present invention are described in details. However, they are only used as examples, and the present invention is not limited to the specific embodiments as described above. For those skilled in the art, any equivalent modifications and substitutions made to the present invention are encompassed in the scope of the present invention. Therefore, all the equal transformations and modifications without departing from the spirit and scope of the present invention should be covered in the scope of the present invention.

What is claimed is:

1. A chimeric antigen receptor comprising a third signal receptor, wherein said chimeric antigen receptor comprises a structure of X-Y-CD3zeta-M-N;
wherein, X comprises a tumor-targeting antibody or a ligand or receptor capable of specifically binding to a tumor;
Y is an intracellular domain of a costimulatory receptor, said costimulatory receptor is selected from ICOS, CD28, CD27, HVEM, LIGHT, CD40L, 4-1BB, OX40, DR3, GITR, CD30, TIM1, SLAM, CD2, CD226;
M is an intracellular domain of a gamma chain family cytokine receptor, said cytokine receptor is selected from IL2Ra, IL2Rb, IL4Ra, IL7Ra, IL9Ra, IL15Ra, IL21Ra; and
N is an intracellular domain of IL2Rg.

2. The chimeric antigen receptor comprising the third signal receptor according to claim 1, wherein said antibody comprises scFv.

3. The chimeric antigen receptor comprising the third signal receptor according to claim 1, wherein said X is selected from anti-CD19 antibody, anti-CD20 antibody, anti-EGFR antibody, anti-HER2 antibody, anti-EGFRVIII antibody, anti-PSMA antibody, anti-BCMA antibody, anti-CD22 antibody, anti-CD30 antibody and anti-CLDN18.2 antibody.

4. The chimeric antigen receptor comprising the third signal receptor according to claim 1, wherein said X is selected from anti-CD19 scFv, anti-CD20 scFv, anti-EGFR scFv, anti-HER2 scFv, anti-EGFRVIII scFv, anti-PSMA scFv, anti-BCMA scFv, anti-CD22 scFv, anti-CD30 scFv and anti-CLDN18.2 scFv.

5. The chimeric antigen receptor comprising the third signal receptor according to claim 4, wherein the sequence of said anti-CD20 scFv is as set forth in SEQ ID No: 1;
the sequence of said anti-CD19 scFv is as set forth in SEQ ID No: 2;
the sequence of said anti-CLDN18.2 scFv is as set forth in SEQ ID No: 3; and/or
the sequence of said anti-EGFR scFv is as set forth in SEQ ID No: 4.

6. The chimeric antigen receptor comprising the third signal receptor according to claim 1, wherein said Y is an intracellular domain of 4-1BB.

7. The chimeric antigen receptor comprising the third signal receptor according to claim 1, wherein the sequence of said intracellular domain of IL7Ra is as set forth in SEQ ID No: 8; the sequence of said intracellular domain of IL2Rb is as set forth in SEQ ID No: 9; the sequence of said intracellular domain of IL4Ra is as set forth in SEQ ID No: 10; the sequence of said intracellular domain of IL9Ra is as set forth in SEQ ID No: 11; the sequence of said intracellular domain of IL21Ra is as set forth in SEQ ID No: 12; the sequence of said intracellular domain of IL2Rg is as set forth in SEQ ID No: 13; the sequence of said intracellular domain of 4-1BB is as set forth in SEQ ID No:6; and/or the sequence of said CD3zata is as set forth in SEQ ID No: 7.

8. The chimeric antigen receptor comprising the third signal receptor according to claim 1, wherein said X is selected from anti-CD19 scFv, anti-CD20 scFv, anti-EGFR scFv, anti-HER2 scFv, anti-EGFRVIII scFv, anti-PSMA scFv, anti-BCMA scFv, anti-CD22 scFv, anti-CD30 scFv and anti-CLDN18.2 scFv;
and said Y is an intracellular domain of 4-1BB, said M is one selected from intracellular domain of IL2Rb, intracellular domain of IL4Ra, intracellular domain of IL7Ra, intracellular domain of IL9Ra, and intracellular domain of IL21Ra.

9. The chimeric antigen receptor comprising the third signal receptor according to claim 1, wherein said X is selected from anti-CD19 scFv, anti-CD20 scFv, anti-EGFR scFv, anti-HER2 scFv, anti-EGFRVIII scFv, anti-PSMA scFv, anti-BCMA scFv, anti-CD22 scFv, anti-CD30 scFv and anti-CLDN18.2 scFv;
and said Y is an intracellular domain of 4-1BB, said M is an intracellular domain of IL7Ra or intracellular domain of IL21Ra.

10. The chimeric antigen receptor comprising the third signal receptor according to claim 1, wherein said chimeric antigen receptor further comprises an extracellular hinge region and a transmembrane domain, said transmembrane domain is selected from CD8a, CD28, CD137 and CD3; said extracellular hinge region is selected from CD8a or IgG.

11. The chimeric antigen receptor comprising the third signal receptor according to claim 10, wherein said extracellular hinge region and transmembrane domain are derived from the extracellular hinge region and transmembrane domain of CD8a,

12. The chimeric antigen receptor comprising the third signal receptor according to claim 11, wherein the sequence of said extracellular hinge region and transmembrane domain is as set forth in SEQ ID No: 5.

13. The chimeric antigen receptor comprising the third signal receptor according to claim 1, wherein said chimeric antigen receptor has a structure of X-H-TM-Y-CD3zeta-M-N;
wherein, X comprises a tumor-targeting antibody or a ligand or receptor capable of specifically binding to a tumor;
H is an extracellular hinge region, said extracellular hinge region is selected from CD8a or IgG;
TM is a transmembrane domain, said transmembrane domain is selected from CD8a, CD28, CD137 and CD3;
Y is an intracellular domain of a costimulatory receptor, said costimulatory receptor is selected from ICOS, CD28, CD27, HVEM, LIGHT, CD40L, 4-1BB, OX40, DR3, GITR, CD30, TIM1, SLAM, CD2, CD226;
M is an intracellular domain of a gamma chain family cytokine receptor, said cytokine receptor is selected from IL2Ra, IL2Rb, IL4Ra, IL7Ra, IL9Ra, IL15Ra, IL21Ra; and
N is an intracellular domain of IL2Rg.

14. The chimeric antigen receptor comprising the third signal receptor according to claim 13, wherein said chimeric antigen receptor has a structure of X-H-TM-Y-CD3zeta-M-N;
wherein, X is selected from anti-CD19 scFv, anti-CD20 scFv, EGFR scFv, HER2 scFv, EGFRVIII scFv, anti-PSMA scFv, anti-BCMA scFv, anti-CD22 scFv, anti-CD30 scFv and anti-CLDN18.2 scFv;
H is an extracellular hinge region of CD8a;
TM is a transmembrane domain of CD8a;
Y is an intracellular domain of 4-1BB;
M is an intracellular domain of a gamma chain family cytokine receptor, said cytokine receptor is selected from IL2Ra, IL2Rb, IL4Ra, IL7Ra, IL9Ra, IL15Ra, IL21Ra; and
N is an intracellular domain of IL2Rg.

15. The chimeric antigen receptor comprising the third signal receptor according to claim 13, wherein the sequence of said H-TM-Y-CD3zeta-M-N is as set forth in any one of SEQ ID No: 23-27.

16. The chimeric antigen receptor comprising the third signal receptor according to claim 13, wherein said X is anti-CD20 scFv.

17. The chimeric antigen receptor comprising the third signal receptor according to claim 16, wherein the sequence of said X-H-TM-Y-CD3zeta is set forth in SEQ ID NO: 15, and/or the sequence of said X-H-TM-Y-CD3zeta-M-N is set forth in SEQ ID NO: 16.

18. A expression vector, comprising the polynucleotide encoding the chimeric antigen receptor comprising the third signal receptor according to claim 1.

19. A chimeric antigen receptor-T (CAR-T) cell comprising an expression vector encoding and expressing the CAR of claim 1.

20. A method of preventing or treating a tumor, comprising administrating said CAR-T cell according to claim 19 to a subject in need thereof.


SEQUENCE LISTING (31 sequences)

SEQ ID NO: 1 - protein, 241 aa, synthetic construct
  REGION 1..241: note = anti-CD20 scFv
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKGGGGSGGGGSGGGGSQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVS

SEQ ID NO: 2 - protein, 242 aa, synthetic construct
  REGION 1..242: note = anti-CD19 scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 3 - protein, 249 aa, synthetic construct
  REGION 1..249: note = anti-CLDN18.2 scFv
DIVMTQSPDSLAVSLGERATISCKSSQSLLNSGNQKNYLTWYQQKPGQPPKLLIYWASTRESGVPDRFSGSGSGTDFTLTISSLQAEDVAVYYCQNDYFYPFTFGQGTKLEIKRTVGGGGSGGGGSGGGGSQVQLVQSGAEVKKPGSSVKVSCKASGYAFSNYLIEWVKQAPGQGLEWIGLINPGSGGTNYNEKFKGKATITADKSTSTAYMELSSLRSEDTAVYYCARVYYGNSFAYWGQGTLVTVSS

SEQ ID NO: 4 - protein, 241 aa, synthetic construct
  REGION 1..241: note = anti-EGFR scFv
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAGGGGSGGGGSGGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 5 - protein, 69 aa, synthetic construct
  REGION 1..69: note = CD8a extracellular hinge region and transmembrane domain
TTTPAPRPPTPAPTIASQPLSLRPEACRPAAGGAVHTRGLDFACDIYIWAPLAGTCGVLLLSLVITLYC

SEQ ID NO: 6 - protein, 42 aa, synthetic construct
  REGION 1..42: note = intracellular domain of 4-1BB
KRGRKKLLYIFKQPFMRPVQTTQEEDGCSCRFPEEEEGGCEL

SEQ ID NO: 7 - protein, 112 aa, synthetic construct
  REGION 1..112: note = intracellular domain of CD3zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 195 aa, synthetic construct
  REGION 1..195: note = intracellular domain of IL7Ra
KKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 9 - protein, 286 aa, synthetic construct
  REGION 1..286: note = intracellular domain of IL2Rb
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 10 - protein, 569 aa, synthetic construct
  REGION 1..569: note = intracellular domain of IL4Ra
KIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS

SEQ ID NO: 11 - protein, 230 aa, synthetic construct
  REGION 1..230: note = intracellular domain of IL9Ra
KLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 12 - protein, 285 aa, synthetic construct
  REGION 1..285: note = intracellular domain of IL21Ra
SLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 13 - protein, 86 aa, synthetic construct
  REGION 1..86: note = intracellular domain of IL2Rg
ERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 14 - protein, 22 aa, synthetic construct
  REGION 1..22: note = signal peptide
METDTLLLWVLLLWVPGSTGTG

SEQ ID NO: 15 - protein, 468 aa, synthetic construct
  REGION 1..468: note = anti-CD20 scFv (X)-H-TM-Y-CD3zeta
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKGGGGSGGGGSGGGGSQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 749 aa, synthetic construct
  REGION 1..749: note = anti-CD20 scFv (X)-H-TM-Y-CD3zeta-M-N
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKGGGGSGGGGSGGGGSQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 17 - protein, 468 aa, synthetic construct
  REGION 1..468: note = anti-CD19 scFv (X)-H-TM-Y-CD3zeta
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - protein, 749 aa, synthetic construct
  REGION 1..749: note = anti-CD19 scFv (X)-H-TM-Y-CD3zeta-M-N
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 19 - protein, 475 aa, synthetic construct
  REGION 1..475: note = anti-CLDN18.2 scFv (X)-H-TM-Y-CD3zeta
DIVMTQSPDSLAVSLGERATISCKSSQSLLNSGNQKNYLTWYQQKPGQPPKLLIYWASTRESGVPDRFSGSGSGTDFTLTISSLQAEDVAVYYCQNDYFYPFTFGQGTKLEIKRTVGGGGSGGGGSGGGGSQVQLVQSGAEVKKPGSSVKVSCKASGYAFSNYLIEWVKQAPGQGLEWIGLINPGSGGTNYNEKFKGKATITADKSTSTAYMELSSLRSEDTAVYYCARVYYGNSFAYWGQGTLVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - protein, 756 aa, synthetic construct
  REGION 1..756: note = anti-CLDN18.2 scFv (X)-H-TM-Y-CD3zeta-M-N
DIVMTQSPDSLAVSLGERATISCKSSQSLLNSGNQKNYLTWYQQKPGQPPKLLIYWASTRESGVPDRFSGSGSGTDFTLTISSLQAEDVAVYYCQNDYFYPFTFGQGTKLEIKRTVGGGGSGGGGSGGGGSQVQLVQSGAEVKKPGSSVKVSCKASGYAFSNYLIEWVKQAPGQGLEWIGLINPGSGGTNYNEKFKGKATITADKSTSTAYMELSSLRSEDTAVYYCARVYYGNSFAYWGQGTLVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 21 - protein, 467 aa, synthetic construct
  REGION 1..467: note = anti-EGFR scFv (X)-H-TM-Y-CD3zeta
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAGGGGSGGGGSGGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 748 aa, synthetic construct
  REGION 1..748: note = anti-EGFR scFv (X)-H-TM-Y-CD3zeta-M-N
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAGGGGSGGGGSGGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 23 - protein, 504 aa, synthetic construct
  REGION 1..504: note = CD8(hinge/TM)-4-BB-CD3zeta-IL7Ra-IL2Rg
TTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 24 - protein, 594 aa, synthetic construct
  REGION 1..594: note = CD8(hinge/TM)-4-BB-CD3zeta-IL21Ra-IL2Rg
TTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQASERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 25 - protein, 595 aa, synthetic construct
  REGION 1..595: note = CD8(hinge/TM)-4-BB-CD3zeta-IL2Rb-IL2Rg
TTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRNCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLVERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 26 - protein, 878 aa, synthetic construct
  REGION 1..878: note = CD8(hinge/TM)-4-BB-CD3zeta-IL4Ra-IL2Rg
TTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRKIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVSERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 27 - protein, 539 aa, synthetic construct
  REGION 1..539: note = CD8(hinge/TM)-4-BB-CD3zeta-IL9Ra-IL2Rg
TTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTFERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 28 - DNA, 789 bp, synthetic construct
  misc_feature 1..789: note = A polynucleotide encoding anti-CD20 scFv
  source 1..789: mol_type = other DNA
atggagaccgacaccctcttattatgggtgctgttattatgggtccccggaagcaccggaaccggtcagatcgtgctgagccagagccccgccattctgtccgccagccccggcgagaaggtgaccatgacatgcagagcctcctcctccgtgagctacatccactggttccagcagaagcccggctcctcccctaagccttggatctacgccaccagcaatctggctagcggagtgcccgtgagattcagcggcagcggaagcggaaccagctactctctgaccatcagcagagtggaggccgaggacgccgctacctactactgtcagcagtggaccagcaaccctcctaccttcggcggcggcaccaagctggagatcaagggcggaggaggctccggaggtggaggttctggcggtggaggttcccaagtgcagctgcagcagcccggcgctgagctggtgaaacccggcgcttccgtgaagatgagctgcaaggccagcggctacaccttcaccagctacaacatgcactgggtgaagcagacccccggcagaggactggaatggatcggcgccatttaccccggcaacggcgatacctcctacaaccagaagttcaagggcaaggctacactgaccgccgacaagagcagcagcaccgcctacatgcagctgagctctctgaccagcgaggacagcgccgtgtactactgcgctagaagcacctactacggcggcgactggtacttcaacgtgtggggagccggcacaaccgtgacagtgtcc

SEQ ID NO: 29 - DNA, 207 bp, synthetic construct
  misc_feature 1..207: note = A polynucleotide encoding the extracellular hinge region and transmembrane domain of CD8a
  source 1..207: mol_type = other DNA
acaaccacccccgctcccagacctcctacacccgctcccaccattgccagccagcctctctctttaagacccgaggcttgtaggcccgctgctggaggagccgtgcacacaaggggactggactttgcttgtgatatctatatctgggcccctctggctggaacttgtggagtcctcttattatctttagtgatcactttatactgt

SEQ ID NO: 30 - DNA, 126 bp, synthetic construct
  misc_feature 1..126: note = A polynucleotide encoding the intracellular domain of 4-1BB
  source 1..126: mol_type = other DNA
aagaggggtcgtaagaagttattatacatcttcaagcagcccttcatgaggcccgtccaaaccacccaagaagaggacggatgtagctgtaggtttcccgaggaggaggagggaggctgcgaatta

SEQ ID NO: 31 - DNA, 336 bp, synthetic construct
  misc_feature 1..336: note = A polynucleotide encoding intracellular domain of CD3zeta
  source 1..336: mol_type = other DNA
cgtgtcaagttctccagaagcgccgatgcccccgcttaccaacaaggtcagaaccagctgtacaatgagctgaatctgggcagaagagaagagtacgacgtgctggataagaggaggggtcgtgaccccgaaatgggaggcaagcccagaagaaaaaacccccaagaaggactctacaacgagctgcaaaaggataagatggctgaggcctattccgagattggcatgaagggcgagagaaggagaggcaagggccacgacggtttatatcaaggtctctccaccgccaccaaggacacatacgatgctctgcacatgcaagctctgccccccaga